(19) United States
(12) Patent Application Publication
(10) Pub. No.: US 2019/0137515 A1
(43) Pub. Date: May 9, 2019

(54) METHODS FOR ESTIMATING MISFOLDED PROTEIN CONCENTRATION IN FLUIDS AND TISSUE BY QUANTITATIVE PMCA

(71) Applicants: Amprion, Inc., San Francisco, CA (US); BOARD OF REGENTS OF THE UNIVERSITY OF TEXAS SYSTEM, Austin, TX (US)

(72) Inventors: Claudio Soto, Friendswood, TX (US); Baian Chen, Beijing (CN); Rodrigo Morales, Houston, TX (US)

(21) Appl. No.: 16/233,848

(22) Filed: Dec. 27, 2018

Related U.S. Application Data
(63) Continuation-in-part of application No. 13/110,899, filed on May 18, 2011, now Pat. No. 10,215,763.
Application No. 13/110,899 is a continuation-in-part of application No. 15/915,554, filed on Mar. 8, 2018.
Application No. 15/915,554 is a continuation of application No. 15/912,552, filed on Mar. 5, 2018, now abandoned.
Application No. 15/912,552 is a continuation of application No. 14/852,471, filed on Sep. 11, 2015, now Pat. No. 9,910,049.
Application No. 14/852,471 is a continuation of application No. 14/852,478, filed on Sep. 11, 2015, now abandoned.
Application No. 14/852,478 is a continuation-in-part of application No. 14/852,475, filed on Sep. 11, 2015.
Application No. 14/852,475 is a continuation-in-part of application No. 15/981,449, filed on May 16, 2018.
(60) Provisional application No. 61/345,940, filed on May 18, 2010.
(60) Provisional application No. 61/345,760, filed on May 18, 2010.
(60) Provisional application No. 62/049,303, filed on Sep. 11, 2014.
(60) Provisional application No. 62/049,306, filed on Sep. 11, 2014.
(60) Provisional application No. 62/049,304, filed on Sep. 11, 2014.
(60) Provisional application No. 62/507,166, filed on May 16, 2017.

(51) Int. Cl. G01N 33/68

(52) U.S. Cl. CPC G01N 33/6896; G01N 2333/4709; G01N 2800/2821; G01N 2800/52

(57) ABSTRACT

Described are methods for estimating misfolded protein concentration in fluids and tissues by quantitative PMCA.

### CROSS-REFERENCE TO RELATED Aβ PLICATIONS

This application is a continuation-in-part of U.S. patent application Ser. No. 13/110,899, filed May 18, 2011, which claims priority to U.S. Provisional Pat. App. Nos. 61/345,940, filed May 18, 2010, and 61/345,760, filed May 18, 2010. This application is also a continuation in part of U.S. patent application Ser. No. 15/915,554, filed Mar. 8, 2018, which is a continuation of U.S. patent application Ser. No. 15/912,552, filed Mar. 5, 2018, which is a continuation of U.S. patent application Ser. Nos. 14/852,471 and 14/852,478, both filed Sep. 11, 2015, which respectively claim priority to U.S. Provisional Pat. App. Nos. 62/049,303 and 62/049,306, both filed Sep. 11, 2014. This application is also a continuation in part of U.S. patent application Ser. No. 14/852,475, filed Sep. 11, 2015, which claims priority to U.S. Provisional Pat. App. No. 62/049,304, filed Sep. 11, 2014. This application is also a continuation in part of U.S. patent application Ser. No. 15/981,449 filed May 16, 2018, which claims priority to U.S. Provisional Pat. App. No. 62/507,166, filed May 16, 2017. The entire contents of the preceding applications are incorporated by reference herein.

### STATEMENT REGARDING FEDERALLY SPONSORED RESEARCH OR DEVELOPMENT

This invention was made with Government support under R01NS049173 and P01AI077774 awarded by the National Institutes of Health. The Government has certain rights in the invention.

### BACKGROUND

Protein misfolding diseases (PMDs) include, for example: prion diseases, amyloidopathies such as Alzheimer's disease (AD), tauopathies such as Parkinson's disease (PD) and AD; synucleopathies such as PD; and the like. Many PMDs represent significant neurodegenerative diseases that affect humans and animals. For example, Creutzfeldt-Jakob disease (CJD), kuru, Gerstmann-Straussler-Scheiker diseases (GSS), and fatal familial insomnia (FFI) in humans, as well as scrapie and bovine spongiform encephalopathy (BSE) in animals, are examples of prion-based transmissible spongiform encephalopathies (TSE). Generally, PMDs are now understood to involve autocatalytic transformation by the misfolded protein in question using the non-misfolded isoform of the corresponding protein as a substrate

A defining characteristic and marker of PMDs is the formation of an abnormally shaped, misfolded protein, for example, in the case of prion diseases, PrP^{Sc}. However, PMDs are characterized by an extremely long incubation period. Thus, the concentration of the corresponding misfolded protein, e.g., PrP^{Sc} in prion diseases, may be at low levels for a long period of time. As such, an objective of PMD research and treatment is to detect small amounts of the corresponding misfolded protein in diverse samples. In addition to detection, it is desirable to quantify the amounts of the corresponding misfolded protein. However, quantification of small amounts of proteins may be difficult, particularly in samples that include biological fluids. Moreover, because small variations in initial conditions may be magnified by exponential processes such as autocatalysis, quantification of small amounts of misfolded proteins using the autocatalytic misfolding reaction may be difficult.

The present application appreciates that quantitatively estimating the amount of misfolded protein in a biological sample may be a challenging endeavor.

### SUMMARY

In one embodiment, a method for preparing a calibration curve useful for quantitatively estimating a concentration of a misfolded protein in a sample is provided. The method may include preparing a plurality of stock solutions. Each stock solution in the plurality of stock solutions may have a known different concentration of the misfolded protein. The method may include separately mixing each of the plurality of stock solutions with a misfolding protein substrate that corresponds to the misfolded protein to form a plurality of separate stock reaction mixes. The method may include forming a plurality of separate amplified portions of the misfolded protein by performing a plurality of protein misfolding cyclic amplification (PMCA) cycles on each of the plurality of separate stock reaction mixes to form a plurality of separate amplified stock reaction mixes comprising the plurality of separate amplified portions of the misfolded protein. Each cycle in the plurality of PMCA cycles may include incubating each stock reaction mix. Each cycle in the plurality of PMCA cycles may include disaggregating aggregates formed in each stock reaction mix. The method may include subjecting each of the plurality of separate amplified stock reaction mixes to an assay for a number of cycles of the plurality of PMCA cycles until a signal of the misfolded protein is detected. The method may include determining the calibration curve according to the known different concentration of the misfolded protein in each stock solution with the number of PMCA cycles corresponding to detection of the signal of the misfolded protein. At least a portion of the known different concentrations of the misfolded protein among the plurality of stock solutions may be below a concentration detectable by the assay such that the calibration curve provides for quantitative estimation of the misfolded protein concentration in the sample below the concentration detectable by the assay. The method may provide that the misfolded protein and the misfolding protein substrate exclude prion protein and isoforms or conformers thereof.

In another embodiment, a method for quantitatively estimating a concentration of a misfolded protein in a sample is provided. The method may include mixing the sample with a misfolding protein substrate to form a reaction mix. The method may include forming an amplified portion of the misfolded protein by performing a plurality of protein misfolding cyclic amplification (PMCA) cycles on the reaction mix to form an amplified reaction mix comprising the amplified portion of the misfolded protein. Each cycle in the plurality of PMCA cycles may include incubating the reaction mix. Each cycle in the plurality of PMCA cycles may include disaggregating aggregates formed in the reaction mix. The method may include subjecting the amplified reaction mix to an assay for a number of the plurality of PMCA cycles until a signal of the misfolded protein is detected. The method may include quantitatively estimating the concentration of the misfolded protein in the sample according to the number of PMCA cycles corresponding to detection of the signal of the misfolded protein by using a predetermined calibration curve for quantitatively estimating the concentration of the misfolded protein in the sample according to the assay. The predetermined calibration curve may be determined according to a plurality of known different concentrations of the misfolded protein each corresponding to a calibrating number of PMCA cycles. Each calibrating number of PMCA cycles may be effective to amplify each corresponding known different concentration of the misfolded protein in the presence of a misfolding protein substrate to a concentration of the misfolded protein detectable by the assay. At least a portion of the plurality of known different concentrations of the misfolded protein may be below the concentration detectable by the assay such that the predetermined calibration curve provides for quantitative estimation of the misfolded protein concentration in the sample below the concentration detectable by the assay. The method may provide that the misfolded protein and the misfolding protein substrate exclude prion protein and isoforms or conformers thereof.

In one embodiment, a kit for quantitatively estimating a concentration of a misfolded protein in a sample is provided. The kit may include a buffer solution that includes at least one misfolding protein substrate. The kit may include at least one predetermined calibration curve for quantitatively estimating the concentration of the at least one misfolded protein in the sample according to an assay. The predetermined calibration curve may be determined according to a plurality of known different concentrations of the at least one misfolded protein each corresponding to a calibrating number of PMCA cycles. Each calibrating number of PMCA cycles may be effective to amplify each corresponding known different concentration of the misfolded protein in the presence of a misfolding protein substrate to a concentration of the misfolded protein detectable by the assay. At least a portion of the plurality of known different concentrations of the misfolded protein may be below the concentration detectable by the assay such that the predetermined calibration curve provides for quantitative estimation of the misfolded protein concentration in the sample below the concentration detectable by the assay. The kit may include instructions. The instructions may direct a user to mix the sample with the buffer solution that includes the at least one misfolding protein substrate to form a reaction mix. The instructions may direct a user to form an amplified portion of the misfolded protein by performing a plurality of protein misfolding cyclic amplification (PMCA) cycles on the reaction mix to form an amplified reaction mix comprising the amplified portion of the misfolded protein. Each cycle in the plurality of PMCA cycles may include incubating the reaction mix. Each cycle in the plurality of PMCA cycles may include disaggregating aggregates formed in the reaction mix. The instructions may direct a user to subject the amplified reaction mix to an assay for a number of the plurality of PMCA cycles until a signal of the misfolded protein is detected. The instructions may direct a user to quantitatively estimate the concentration of the misfolded protein in the sample according to the number of PMCA cycles corresponding to detection of the signal of the misfolded protein by using the predetermined calibration curve.

### BRIEF DESCRIPTION OF THE DRAWINGS

The accompanying figures, which are incorporated in and constitute a part of the specification, illustrate various methods, results, and so on, and are used merely to illustrate various example embodiments.

FIG. 1 illustrates an example schematic representation of the conversion of PrP ^{C} to PrP^{Sc}.

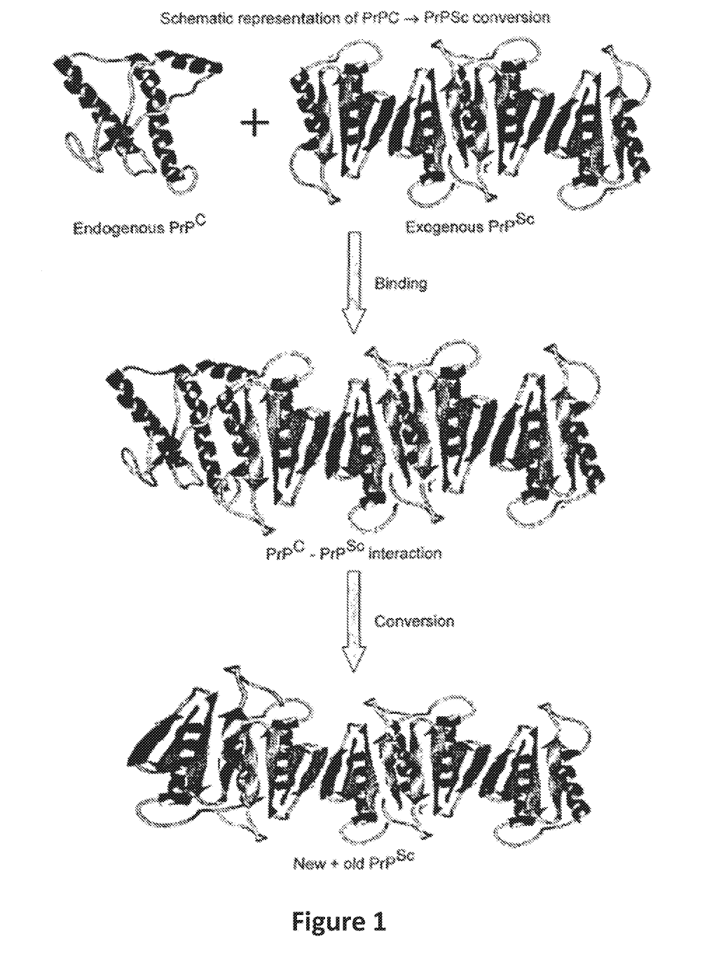

FIG. 2 illustrates an example diagrammatic representation of the protein misfolding amplification procedure.

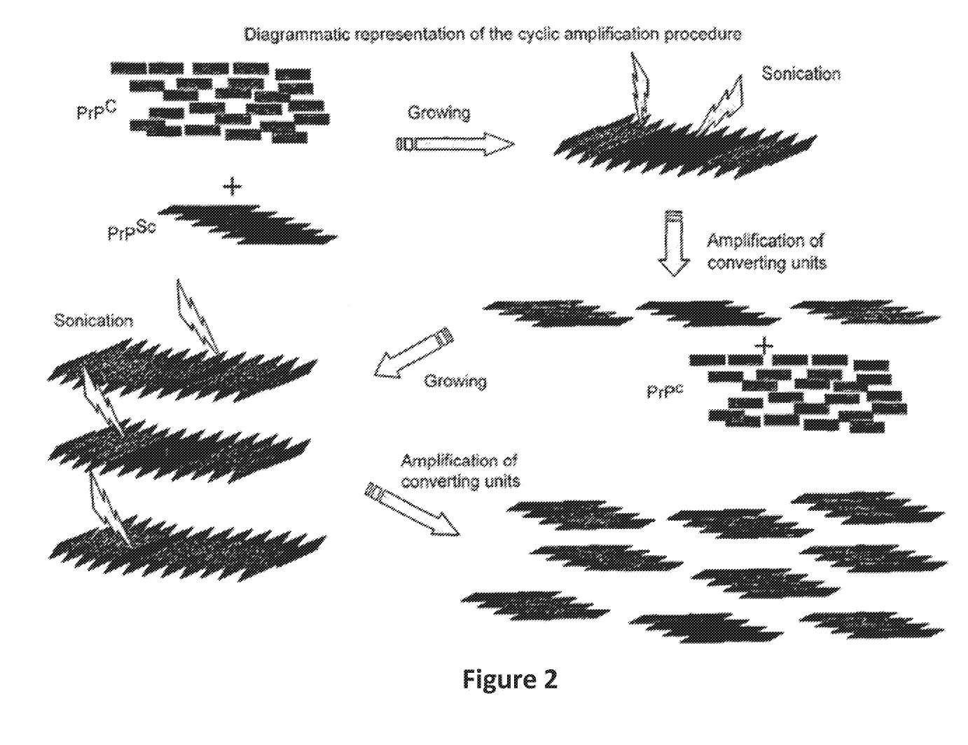

FIG. 3a illustrates western blot assays (3F4 antibody) of stock solutions of PrP^{Sc} of various concentrations, upon being subjected to normal brain homogenate and serial rounds of PMCA (144 cycles).

FIG. 3b illustrates a calibration curve, based on a plot of PrP^{Sc} concentration vs. the number of PMCA rounds required to detect PrP^{Sc} by western blot assay.

FIG. 4 is a flow chart of an example method for estimating the concentration of prion in a sample, where a predetermined calibration curve is provided.

FIG. 5 illustrates western blot assays of PrP^{Sc}-affected hamster spleen suspended in normal hamster brain homogenate and subjected to serial PMCA, as compared to various control samples.

FIG. 6 illustrates western blot assays of PrP^{Sc}-affected hamster spleen suspended in normal hamster brain homogenate and subjected to serial PMCA. The samples were taken at various time periods after the hamsters were inoculated with PrP^{Sc}.

FIG. 7 illustrates plots of concentrations of PrP^{Sc} in PrP^{Sc}-affected hamster spleen, brain, buffy coat, and plasma after serial rounds of PMCA. The samples were taken at various time periods after the hamsters were inoculated with PrP^{Sc}.

### DETAILED DESCRIPTION

Described herein in various embodiments are a method for determining a calibration curve for estimating a misfolded concentration in fluids and tissues by quantitative PMCA, a method for using the calibration curve for estimating a misfolded concentration in fluids and tissues by quantitative PMCA, and a kit for for using the calibration curve for estimating a misfolded concentration in fluids and tissues by quantitative PMCA.

In one embodiment, a method for preparing a calibration curve useful for quantitatively estimating a concentration of a misfolded protein in a sample is provided. The method may include preparing a plurality of stock solutions. Each stock solution in the plurality of stock solutions may have a known different concentration of the misfolded protein. The method may include separately mixing each of the plurality of stock solutions with a misfolding protein substrate that corresponds to the misfolded protein to form a plurality of separate stock reaction mixes. The method may include forming a plurality of separate amplified portions of the misfolded protein by performing a plurality of protein misfolding cyclic amplification (PMCA) cycles on each of the plurality of separate stock reaction mixes to form a plurality of separate amplified stock reaction mixes comprising the plurality of separate amplified portions of the misfolded protein. Each cycle in the plurality of PMCA cycles may include incubating each stock reaction mix. Each cycle in the plurality of PMCA cycles may include disaggregating aggregates formed in each stock reaction mix. The method may include subjecting each of the plurality of separate amplified stock reaction mixes to an assay for a number of cycles of the plurality of PMCA cycles until a signal of the misfolded protein is detected. The method may include determining the calibration curve according to the known different concentration of the misfolded protein in each stock solution with the number of PMCA cycles corresponding to detection of the signal of the misfolded protein. At least a portion of the known different concentrations of the misfolded protein among the plurality of stock solutions may be below a concentration detectable by the assay such that the calibration curve provides for quantitative estimation of the misfolded protein concentration in the sample below the concentration detectable by the assay. The method may provide that the misfolded protein and the misfolding protein substrate exclude prion protein and isoforms or conformers thereof.

As used herein, the term “incubation mixture” encompasses the terms “stock reaction mix” and “reaction mix” as used in a PMCA reaction. As used herein, references to determining the presence of a misfolded protein, or detecting a misfolded protein may also refer to determining the amount of the misfolded protein according to the methods described herein, for example, using the calibration curve.

As used herein, a “misfolding protein substrate” is a non-misfolded isoform of the misfolded protein. The misfolding protein substrate may be in a native form, e.g., in a native folded conformation, a native unfolded, soluble conformation, a native folded, soluble conformation, and the like. The misfolding protein substrate may be a non-pathological isoform. The misfolding protein substrate may be a monomer. In some embodiments, the misfolding protein substrate may be an oligomer or polymer of monomeric repeat units.

The misfolding protein substrate or a repeat unit thereof may each have a sequence corresponding to the misfolded protein or a repeat unit thereof

The misfolding protein substrate and misfolded protein may together be capable, under corresponding PMCA incubation conditions, of causing the misfolding protein substrate to adopt the isoform of the misfolded protein to form an additional amount of the misfolded protein. Exemplary corresponding pairs of misfolding protein substrate and misfolded protein may include, for example: natively folded Aβ and misfolded Aβ; unfolded αSynuclein and misfolded αSynuclein; tau and misfolded tau, e.g., 4R tau and misfolded 4R tau, 3R tau and misfolded 3R tau; and the like. In some embodiments, the misfolding protein substrate and the misfolded protein may exclude 3R tau protein. In some embodiments, the misfolded protein may be soluble. The misfolded protein may exclude insoluble misfolded protein. The misfolded protein may exclude insoluble deposits, plaques, and fibrils.

In some embodiments, the misfolding protein substrate and the misfolded protein may exclude prion protein and/or isoforms, oligomers, polymers, aggregates, seeds, deposits, plaques, fibrils, soluble forms, and/or insoluble forms thereof. For example, the misfolding protein substrate may exclude PrP^{C}. The misfolded protein may exclude PrP^{Sc}. The misfolded protein may exclude PrP^{Res}.

In some embodiments, disaggregating in the PMCA cycles may include physically disrupting the reaction mix. Physically disrupting the reaction mix may include one or more of: sonication, stirring, shaking, freezing/thawing, laser irradiation, autoclave incubation, high pressure, and homogenization. For example, shaking may include cyclic agitation. The cyclic agitation may be conducted between about 50 rotations per minute (RPM) and 10,000 RPM. The cyclic agitation may be conducted between about 200 RPM and about 2000 RPM. The cyclic agitation may be conducted at about 500 RPM.

In various embodiments, the physically disrupting the reaction mix may be conducted in each incubation cycle for between about 5 seconds and about 10 minutes, between about 30 sec and about 1 minute, between about 45 sec and about 1 minute, for about 1 minute, and the like. For example, the physically disrupting the reaction mix may be conducted in each incubation cycle by shaking for one or more of: between about 5 seconds and about 10 minutes, between about 30 sec and about 1 minute, between about 45 sec and about 1 minute, for about 1 minute, and the like. The incubating the reaction mix may be independently conducted, in each incubation cycle, for a time between about 1 minutes and about 5 hours, between about 10 minutes and about 2 hours, between about 15 minutes and about 1 hour, between about 25 minutes and about 45 minutes, and the like. Each incubation cycle may include independently incubating and physically disrupting the reaction mix for one or more of: incubating between about 1 minutes and about 5 hours and physically disrupting between about 5 seconds and about 10 minutes; incubating between about 10 minutes and about 2 hours and physically disrupting between about 30 sec and about 1 minute; incubating between about 15 minutes and about 1 hour and physically disrupting between about 45 sec and about 1 minute; incubating between about 25 minutes and about 45 minutes and physically disrupting between about 45 sec and about 1 minute; and incubating about 1 minute and physically disrupting about 1 minute.

The conducting the incubation cycle may be repeated between about 2 times and about 1000 times, between about 5 times and about 500 times, between about 50 times and about 500 times, between about 150 times and about 250 times, and the like. The incubating the reaction mix being independently conducted, in each incubation cycle, at a temperature in ° C. of about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range between any two of the preceding values, for example, between about 15° C. and about 50° C.

In some embodiments, the method may include plotting the calibration curve in the form of a standard calibration curve.

In several embodiments of the method, the misfolding protein substrate may be provided for mixing with each of the plurality of stock solutions in the form of a normal tissue homogenate that includes the misfolding protein substrate. The misfolding protein substrate may be provided as a normal biological fluid that includes the misfolding protein substrate. The misfolding protein substrate may be purified from one or more of the normal tissue homogenate and the normal biological fluid. The misfolding protein substrate may be a recombinant preparation of the misfolding protein substrate.

In various embodiments, detecting the misfolding protein substrate i may include one or more of: a Western Blot assay, a dot blot assay, an enzyme-linked immunosorbent assay (ELISA), a thioflavin T binding assay, a Congo Red binding assay, a sedimentation assay, electron microscopy, atomic force microscopy, surface plasmon resonance, spectroscopy, and the like. The ELISA may include a two-sided sandwich ELISA. The spectroscopy may include one or more of: quasi-light scattering spectroscopy, multispectral ultraviolet spectroscopy, confocal dual-color fluorescence correlation spectroscopy, Fourier-transform infrared spectroscopy, capillary electrophoresis with spectroscopic detection, electron spin resonance spectroscopy, nuclear magnetic resonance spectroscopy, Fluorescence Resonance Energy Transfer (FRET) spectroscopy, and the like.

For example, the assay may be one of: a western blot assay and a fluorescence assay. Determining the presence of the misfolding protein substrate in the sample may include detecting the indicating state of the indicator of the misfolding protein substrate in the detection mixture. The indicating state of the indicator and the non-indicating state of the indicator may be characterized by a difference in fluorescence, light absorption or radioactivity depending on the specific indicator. Determining the presence of the misfolding protein substrate in the sample may include detecting the difference in these parameters.

In several embodiments, the method may include contacting a molar excess of the indicator of the misfolding protein substrate to one or both of the reaction mix or the detection mixture. The molar excess may be greater than a total molar amount of protein monomer included in the misfolding protein substrate in the reaction mix.

In various embodiments, the indicator of the misfolding protein substrate may include one or more of: Thioflavin T, Congo Red, m-I-Stilbene, Chrysamine G, PIB, BF-227, X-34, TZDM, FDDNP, MeO-X-04, IMPY or NIAD-4, luminescent conjugated polythiophenes, a fusion with a fluorescent protein such as green fluorescent protein and yellow fluorescent protein, derivatives thereof, and the like.

In various embodiments, the detecting the misfolding protein substrate in the detection mixture may include contacting the reaction mix with a protease. The misfolding protein substrate may be detected in the detection mixture using sequence-based or anti-misfolded protein antibodies in one or more of: a Western Blot assay, a dot blot assay, and an ELISA.

In some embodiments, the method may include providing the misfolding protein substrate in labeled form. The monomeric Aβ protein in labeled form may include one or more of: a covalently incorporated radioactive amino acid, a covalently incorporated, isotopically labeled amino acid, and a covalently incorporated fluorophore. The detecting the misfolding protein substrate may include detecting in labeled form as incorporated into the amplified portion of the misfolding protein substrate.

In various embodiments, the method may include preparing the stock reaction mixes including a biological fluid. Preparing the stock reaction mixes including a biological fluid may be effective to provide the calibration curve for quantitatively estimating the concentration of the misfolded protein in a sample comprising the biological fluid. The biological fluid may include, for example, one or more of: amniotic fluid; bile; blood; cerebrospinal fluid; cerumen; skin; exudate; feces; gastric fluid; lymph; milk; mucus; mucosal membrane; peritoneal fluid; plasma; pleural fluid; pus; saliva; sebum; semen; sweat; synovial fluid; tears; and urine.

In some embodiments of the method, the misfolded protein and the misfolding protein substrate may correspond to one of: Aβ; αS; 3R tau; and 4R tau. For example, the misfolded protein may be misfolded Aβ and the misfolding protein substrate may be native, folded Aft In some embodiments, the misfolded protein and the misfolding protein substrate may exclude 3R tau.

In some embodiments, the reaction mix may include the misfolding protein substrate in a concentration, or in a concentration range, of one or more of: between about 1 nM and about 2 mM; between about 10 nM and about 200 μM; between about 100 nM and about 20 μM; or between about 1 μM and about 10 μM; and about 2 μM.

In several embodiments, the reaction mix may include a buffer composition. The buffer composition may be effective to prepare or maintain the pH of the reaction mix as described herein, e.g., between pH 5 and pH 9. The buffer composition may include one or more of: Tris-HCL, PBS, MES, PIPES, MOPS, BES, TES, or HEPES, and the like. The buffer concentration may be at a total concentration of between about 1 μm and about 1M. For example, the buffer may be Tris-HCL at a concentration of 0.1 M.

In various embodiments, the reaction mix may include a salt composition. The salt composition may be effective to increase the ionic strength of the reaction mix. The salt composition may include one or more of: NaCl or KCl, and the like. The reaction mix may include the salt composition at a total concentration of between about 1 μm and about 500 mM.

In several embodiments, the reaction mix may be characterized by, prepared with, or maintained at a pH value of or a pH range of one or more of: between about 5 and about 9; between about 6 and about 8.5; between about 7 and about 8; and about 7.4.

In some embodiments, the reaction mix may be incubated at a temperature in ° C. of about one or more of 4, 6, 8, 10, 12, 14, 16, 18, 20, 22, 24, 26, 28, 30, 32, 34, 35, 36, 37, 40, 45, 50, 55, and 60, e.g., about 22° C., or a temperature range between any two of the preceding values, for example, one or more of: between about 4° C. and about 60° C.; between about 4° C. and about 35° C.; between about 8° C. and about 50° C.; between about 12° C. and about 40° C.; between about 18° C. and about 30° C.; between about 18° C. and about 26° C.; and the like.

In another embodiment, a method for quantitatively estimating a concentration of a misfolded protein in a sample is provided. The method may include mixing the sample with a misfolding protein substrate to form a reaction mix. The method may include forming an amplified portion of the misfolded protein by performing a plurality of protein misfolding cyclic amplification (PMCA) cycles on the reaction mix to form an amplified reaction mix comprising the amplified portion of the misfolded protein. Each cycle in the plurality of PMCA cycles may include incubating the reaction mix. Each cycle in the plurality of PMCA cycles may include disaggregating aggregates formed in the reaction mix. The method may include subjecting the amplified reaction mix to an assay for a number of the plurality of PMCA cycles until a signal of the misfolded protein is detected. The method may include quantitatively estimating the concentration of the misfolded protein in the sample according to the number of PMCA cycles corresponding to detection of the signal of the misfolded protein by using a predetermined calibration curve for quantitatively estimating the concentration of the misfolded protein in the sample according to the assay. The predetermined calibration curve may be determined according to a plurality of known different concentrations of the misfolded protein each corresponding to a calibrating number of PMCA cycles. Each calibrating number of PMCA cycles may be effective to amplify each corresponding known different concentration of the misfolded protein in the presence of a misfolding protein substrate to a concentration of the misfolded protein detectable by the assay. At least a portion of the plurality of known different concentrations of the misfolded protein may be below the concentration detectable by the assay such that the predetermined calibration curve provides for quantitative estimation of the misfolded protein concentration in the sample below the concentration detectable by the assay. The method may provide that the misfolded protein and the misfolding protein substrate exclude prion protein and isoforms or conformers thereof.

In various embodiments, the method for quantitatively estimating a concentration of a misfolded protein in a sample may include any aspect of the method for preparing a calibration curve.

For example, in some embodiments, the misfolding protein substrate may be provided for mixing with each of the plurality of stock solutions in the form of a normal tissue homogenate that includes the misfolding protein substrate. The misfolding protein substrate may be provided as a normal biological fluid that includes the misfolding protein substrate. The misfolding protein substrate may be purified from one or more of the normal tissue homogenate and the normal biological fluid. The misfolding protein substrate may be a recombinant preparation of the misfolding protein substrate.

In some embodiments, the calibration curve may be in the form of a standard calibration curve. The assay may be one of: a western blot assay and a fluorescence assay. The disaggregating may include subjecting the reaction mix to sonication.

In various embodiments, the method may include removing a portion of the reaction mix. The method may include contacting the portion with an additional portion of the misfolding protein substrate to form a second reaction mix. The method may include performing a plurality of PMCA cycles on the second reaction mix. Each cycle in the plurality of PMCA cycles may include incubating the second reaction mix; and disaggregating aggregates formed in the second reaction mix. The method may include subjecting the disaggregated second reaction mix to an assay for a number of cycles of the plurality of PMCA cycles until the signal of the misfolded protein is detected. The method may include quantitatively estimating the concentration of the misfolded protein in the second reaction mix according to the number of cycles corresponding to detection of the signal of the misfolded protein by using the predetermined calibration curve.

In various embodiments, the sample may include a biological fluid including one or more of: amniotic fluid; bile; blood; cerebrospinal fluid; cerumen; skin; exudate; feces; gastric fluid; lymph; milk; mucus; mucosal membrane; peritoneal fluid; plasma; pleural fluid; pus; saliva; sebum; semen; sweat; synovial fluid; tears; and urine. The calibration curve may have been developed in the presence of the biological fluid included in the sample.

In some embodiments, quantitatively estimating the concentration of the misfolded protein in the sample may include quantitatively estimating the concentration of the misfolded protein below the concentration detectable by the assay.

In some embodiments of the method, the misfolded protein and the misfolding protein substrate may correspond to one of: Aβ; αS; 3R tau; and 4R tau. For example, the misfolded protein may be misfolded Aβ and the misfolding protein substrate may be native, folded Aβ. In some embodiments, the misfolded protein and the misfolding protein substrate may exclude 3R tau.

In several embodiments, the sample may include one or more additional misfolded and/or non-misfolded proteins different from the misfolded protein and the misfolding protein substrate.

In various embodiments, the methods may include selectively concentrating the misfolded protein in one or more of the sample and the reaction mix. The selectively concentrating the misfolded protein may include pre-treating the sample prior to forming the reaction mix. The selectively concentrating the misfolded protein may include pre-treating the reaction mix prior to incubating the reaction mix. The selectively concentrating the misfolded protein may include contacting one or more antibodies capable of binding the misfolded protein to form a captured misfolded protein. The one or more antibodies capable of binding the misfolded protein may include one or more of: an antibody specific for an amino acid epitope sequence of the misfolded protein, and an antibody specific for a conformation of the misfolded protein. The antibody specific for a conformation of the misfolded protein may be selective for a conformational epitope of a tauopathy-specific misfolded protein. The one or more one or more antibodies capable of binding the misfolded protein may be coupled to a solid phase. The solid phase may include one or more of a magnetic bead and a multiwell plate.

For example, ELISA plates may be coated with the antibodies used to capture misfolded protein from the patient sample. The antibody-coated ELISA plates may be incubated with a patient sample, unbound materials may be washed off, and the PMCA reaction may be performed. Antibodies may also be coupled to beads. The beads may be incubated with the patient sample and used to separate misfolded protein -antibody complexes from the remainder of the patient sample.

In some embodiments of the methods, the capturing the misfolded protein from the sample to form a captured misfolded protein may be conducted using one or more antibodies specific for the misfolded protein. The one or more antibodies specific for the misfolded protein may include one or more of: an antibody specific for an amino acid epitope sequence of the misfolded protein and an antibody specific for a conformation of the misfolded protein. The antibody specific for a conformation of the misfolded protein may be selective for a conformational epitope of a tauopathy-specific misfolded protein. The antibody specific for the conformation of the misfolded protein may correspond to one of: Alzheimer's disease (AD), Parkinson's Disease (PD), Progressive Supranuclear Palsy (PSP), FrontoTemporal Dementia (FTD), Corticobasal degeneration (CBD), Mild cognitive impairment (MCI), Argyrophilic grain disease (AgD) Traumatic Brain Injury (TBI), Chronic Traumatic Encephalopathy (CTE), and Dementia Pugilistica (DP). The one or more antibodies specific for the misfolded protein may be coupled to a solid phase. The solid phase may include one or more of a magnetic bead and a multiwell plate. Contacting the sample with the misfolding substrate protein to form the reaction mix may include contacting a molar excess of the misfolding substrate protein to the sample. The molar excess of the misfolding substrate protein may be greater than a total molar amount of protein monomer included in the captured misfolded protein. Incubating the reaction mix may be effective to cause misfolding and/or aggregation of the misfolding substrate protein in the presence of the captured misfolded protein to form the amplified misfolded protein. The misfolding substrate protein may include 4R tau protein.

As used herein, “Aβ” or “beta amyloid” refers to a peptide formed via sequential cleavage of the amyloid precursor protein (Aβ P). Various Aβ isoforms may include 38-43 amino acid residues. The Aβ protein may be formed when Aβ P is processed by β- and/or γ-secretases in any combination. The Aβ may be a constituent of amyloid plaques in brains of individuals suffering from or suspected of having AD. Various Aβ isoforms may include and are not limited to Abeta40 and Abeta42. Various Aβ peptides may be associated with neuronal damage associated with AD.

In embodiments where the misfolded protein and the misfolding protein substrate may correspond to Aβ, the methods may include conducting an incubation cycle two or more times on the reaction mix effective to form an amplified portion of misfolded Aβ protein from the monomeric, folded Aβ protein. Each incubation cycle may include incubating the reaction mix effective to cause misfolding and/or aggregation of at least a portion of the monomeric, folded Aβ protein in the presence of the soluble, misfolded Aβ protein. Each incubation cycle may include physically disrupting the reaction mix effective to at least partly de-aggregate at least a portion of a misfolded Aβ aggregate present. The methods may include determining the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded aggregate, and an insoluble, misfolded Aβ aggregate.

As used herein, “monomeric, folded Aβ protein” refers to single Aβ protein molecules in their native, nonpathogenic, folded configuration. “Soluble, misfolded Aβ protein” refers to misfolded monomers or aggregates of Aβ protein that remain in solution. Examples of soluble, misfolded Aβ protein may include any number of aggregated misfolded Aβ protein monomers so long as the misfolded Aβ protein remains soluble. For example, soluble, misfolded Aβ protein may include aggregates of between 2 and about 50 units of misfolded Aβ protein monomer. In some examples, aggregates may be referred to as oligomers or polymers. In some examples, aggregation may be referred to as oligomerization or polymerization.

Soluble, misfolded Aβ protein may aggregate or oligomerize to form insoluble aggregates and/or higher oligomers, leading to Aβ protein aggregates in the form of protofibrils, fibrils, and eventually amyloid plaques. “Seeds” or “nuclei” of Aβ refer to misfolded Aβ protein or short fragmented fibrils, particularly soluble, misfolded Aβ protein, with catalytic activity for inducing further misfolding, oligomerization, and/or aggregation. Such nucleation-dependent polymerization may be characterized by a slow lag phase wherein aggregated nuclei may form, which may then catalyze rapid formation of further and/or larger aggregates. The lag phase may be minimized or removed by addition of pre-formed nuclei or seeds. In some examples, “seeds” or “nuclei” may exclude unaggregated monomers of Aβ protein. Without wishing to be bound by theory, it is believed that at least under some conditions, monomeric, misfolded Aβ protein may not be stable, and the minimum stable size of pathogenic, misfolded Aβ protein may be an aggregate of two monomer units of misfolded Aβ protein.

As used herein, aggregates of Aβ protein refer to non-covalent associations of protein including soluble, misfolded Aβ protein. Aggregates of Aβ protein may be “de-aggregated”, broken up, or disrupted to release smaller aggregates, e.g., soluble, misfolded Aβ protein and fragmented fibrils. The catalytic activity of a collection of misfolded Aβ protein aggregate seeds may scale, at least in part with the number of seeds in a mixture. Accordingly, disruption of aggregates of Aβ protein in a mixture to release soluble, misfolded Aβ protein and fragmented fibrils seeds may lead to an increase in catalytic activity for aggregation of monomeric Aβ protein.

In various embodiments, methods for determining a presence, absence, or amount of a soluble, misfolded Aβ protein in a sample are provided, e.g., for determining the calibration curve or for quantitatively estimating a concentration of a misfolded protein in a sample according to the calibration curve. The methods may include contacting the sample with an indicator, e.g., Thioflavin T, and a monomeric, folded Aβ protein to form a reaction mix. The methods may include conducting an incubation cycle two or more times on the reaction mix effective to form an amplified portion of misfolded Aβ protein from the monomeric, folded Aβ protein. Each incubation cycle may include incubating the reaction mix effective to cause misfolding and/or aggregation of at least a portion of the monomeric, folded Aβ protein in the presence of the soluble, misfolded Aβ protein. Each incubation cycle may include shaking the reaction mix effective to at least partly de-aggregate at least a portion of a misfolded aggregate present. The methods may include determining the presence, absence, or amount of the soluble, misfolded Aβ protein in the sample by detecting a fluorescence of the Thioflavin T corresponding to at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In various embodiments, methods for determining a presence, absence, or amount of a soluble, misfolded Aβ protein in a sample are provided, e.g., for determining the calibration curve or for quantitatively estimating a concentration of a misfolded protein in a sample according to the calibration curve. The methods may include capturing a soluble, misfolded Aβ protein from the sample to form a captured soluble, misfolded Aβ protein. The methods may include contacting the captured, misfolded Aβ protein with a molar excess of monomeric, folded Aβ protein to form a reaction mix. The molar excess may be greater than an amount of Aβ protein monomer included in the captured soluble, misfolded Aβ protein. The methods may include conducting an incubation cycle two or more times on the reaction mix effective to form an amplified portion of misfolded Aβ protein from the monomeric, folded Aβ protein. Each incubation cycle may include incubating the reaction mix effective to cause misfolding and/or aggregation of at least a portion of the monomeric, folded Aβ protein in the presence of the captured soluble, misfolded Aβ protein. Each incubation cycle may include physically disrupting the reaction mix effective to at least partly de-aggregate at least a portion of a misfolded Aβ aggregate present. The methods may include determining the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The captured, soluble, misfolded Aβ protein may include one or more of: a captured, soluble, misfolded Aβ monomer and a captured, soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In some embodiments, the methods may include contacting an indicator of the soluble, misfolded protein to one or both of the reaction mix or the detection mixture. The indicator of the soluble, misfolded Aβ protein may be characterized by an indicating state in the presence of the soluble, misfolded Aβ protein and a non-indicating state in the absence of the soluble, misfolded Aβ protein. In several embodiments, the sample may be taken from a subject. The method may include determining or diagnosing the presence of AD in the subject according to the presence, absence, or amount of the soluble, misfolded Aβ protein in the sample. The presence, absence, or amount of the soluble, misfolded Aβ protein in the sample may be determined compared to a control sample taken from a control subject.

In various embodiments, the detecting may include detecting an amount of the soluble, misfolded Aβ protein in the sample according to the calibration curve. The method may include determining or diagnosing the presence of AD in the subject by comparing the amount of the soluble, misfolded Aβ protein in the sample to the calibration curve as described herein.

In several embodiments, the sample may be taken from a subject exhibiting no clinical signs of dementia according to cognitive testing. The method may include determining or diagnosing the presence of AD in the subject according to the presence, absence, or amount of the soluble, misfolded Aβ protein in the sample.

In some embodiments, the sample may be taken from a subject exhibiting no cortex plaques or tangles according to amyloid beta contrast imaging. The method may further include determining or diagnosing the presence of AD in the subject according to the presence, absence, or amount of the soluble, misfolded Aβ protein in the sample.

In various embodiments, the sample may be taken from a subject exhibiting clinical signs of dementia according to cognitive testing. The method may further include determining or diagnosing the presence of AD as a contributing factor to the clinical signs of dementia in the subject according to the presence, absence, or amount of the soluble, misfolded Aβ protein in the sample.

In several embodiments, the method may include taking the sample from the subject. The subject may be one of a: human, mouse, rat, dog, cat, cattle, horse, deer, elk, sheep, goat, pig, or non-human primate. Non-human animals may be wild or domesticated. The subject may be one or more of: at risk of AD, having AD, and under treatment for AD, at risk of having a disease associated with dysregulation, misfolding, aggregation or disposition of Aβ, having a disease associated with dysregulation, misfolding, aggregation or disposition of Aβ, or under treatment for a disease associated with dysregulation, misfolding, aggregation or disposition of Aβ.

In various embodiments, the method may include determining or diagnosing a progression or homeostasis of AD in the subject by comparing the amount of the soluble, misfolded Aβ protein in the sample to an amount of the soluble, misfolded Aβ protein in a comparison sample taken from the subject at a different time compared to the sample.

For example, several novel therapeutics that are targeting Aβ homeostasis through various mechanisms are currently under development. A PMCA assay for Aβ oligomers may be employed to determine which patients may be treated with an Aβ modulating therapy. Patients showing a change, e.g., decrease or increase, in the level of Aβ oligomers as detected by the PMCA method may be classified as “responders” to Aβ modulating therapy, and may be treated with a therapeutic reducing the levels of Aβ oligomers. Patients lacking an aberrant Aβ homeostasis may be classified as “non responders” and may not be treated. Patients who could benefit from therapies aimed at modulating Aβ homeostasis may thus be identified.

Further, for example, the amount of Aβ oligomers may be measured in samples from patients using PMCA. Patients with elevated Aβ measurements may be treated with therapeutics modulating Aβ homeostasis. Patients with normal Aβ measurements may not be treated. A response of a patient to therapies aimed at modulating Aβ homeostasis may be followed. For example, Aβ oligomer levels may be measured in a patient sample at the beginning of a therapeutic intervention. Following treatment of the patient for a clinical meaningful period of time, another patient sample may be obtained and Aβ oligomer levels may be measured. Patients who show a change in Aβ levels following therapeutic intervention may be considered to respond to the treatment. Patients who show unchanged Aβ levels may be considered non-responding. The methods may include detection of Aβ aggregates in patient samples containing components that may interfere with the PMCA reaction.

In some embodiments, the subject may be treated with an Aβ modulating therapy. The method may include comparing the amount of the soluble, misfolded Aβ protein in the sample to an amount of the soluble, misfolded Aβ protein in a comparison sample. The sample and the comparison sample may be taken from the subject at different times over a period of time under the Aβ modulating therapy. The method may include determining or diagnosing the subject is one of: responsive to the Aβ modulating therapy according to a change in the soluble, misfolded Aβ protein over the period of time, or non-responsive to the Aβ modulating therapy according to homeostasis of the soluble, misfolded Aβ protein over the period of time. The method may include treating the subject determined to be responsive to the Aβ modulating therapy with the Aβ modulating therapy. The Aβ modulating therapy may include administration of one or more of: an inhibitor of BACE1 (beta-secretase 1); an inhibitor of γ-secretase; and a modulator of Aβ homeostasis, e.g., an immunotherapeutic modulator of Aβ homeostasis. The Aβ modulating therapy may include administration of one or more of: E2609; MK-8931; LY2886721; AZD3293; semagacestat (LY-450139); avagacestat (BMS-708163); solanezumab; crenezumab; bapineuzumab; BIIB037; CAD106; 8F5 or 5598 or other antibodies raised against Aβ globulomers, e.g., as described by Barghorn et al, “Globular amyloid β-peptidei₁₋₄₂ oligomer-a homogenous and stable neuropathological protein in Alzheimer's disease” J. Neurochem., 2005, 95, 834-847, the entire teachings of which are incorporated herein by reference; ACC-001; V950; Affitrope AD02; and the like.

In several embodiments, the method may include selectively concentrating the soluble, misfolded Aβ protein in one or more of the sample, the reaction mix, and the detection mixture. The selectively concentrating the soluble, misfolded Aβ protein may include pre-treating the sample prior to forming the reaction mix. The selectively concentrating the soluble, misfolded Aβ protein may include pre-treating the reaction mix prior to incubating the reaction mix. The selectively concentrating the soluble, misfolded Aβ protein may include contacting one or more Aβ specific antibodies to the soluble, misfolded Aβ protein to form a captured soluble, misfolded Aβ protein. The one or more Aβ specific antibodies may include one or more of: 6E10, 4G8, 82E1, A11, X-40/42, and 16ADV. Such antibodies may be obtained as follows: 6E10 and 4G8 (Covance, Princeton, N.J.); 82E1 (IBL America, Minneapolis, Minn.); A11 (Invitrogen, Carlsbad, Calif.); X-40/42 (MyBioSource, Inc., San Diego, Calif.); and 16ADV (Acumen Pharmaceuticals, Livermore, Calif.). The one or more Aβ specific antibodies may include one or more of: an antibody specific for an amino acid sequence of Aβ and an antibody specific for a conformation of the soluble, misfolded Aβ protein. The one or more Aβ specific antibodies may be coupled to a solid phase. The solid phase may include one or more of a magnetic bead and a multiwell plate.

For example, ELISA plates may be coated with the antibodies used to capture Aβ from the patient sample. The antibody-coated ELISA plates may be incubated with a patient sample, unbound materials may be washed off, and the PMCA reaction may be performed. Antibodies may also be coupled to beads. The beads may be incubated with the patient sample and used to separate Aβ-antibody complexes from the remainder of the patient sample.

In various embodiments, the contacting the sample with the monomeric Aβ protein to form the reaction mix may include contacting a molar excess of the monomeric Aβ protein to the sample including the captured soluble, misfolded Aβ protein. The molar excess of the monomeric Aβ protein may be greater than a total molar amount of Aβ protein monomer included in the captured soluble, misfolded Aβ protein. The incubating the reaction mix may be effective to cause oligomerization of at least a portion of the monomeric Aβ protein in the presence of the captured soluble, misfolded Aβ protein to form the amplified portion of the soluble, misfolded Aβ protein.

In some embodiments, the protein aggregate may include one or more of: the monomeric Aβ protein, the soluble, misfolded Aβ protein, a captured form of the soluble, misfolded Aβ protein, larger Aβ aggregates, and the like.

In several embodiments, contacting the sample with the monomeric Aβ protein to form the reaction mix may be conducted under physiological conditions. Contacting the sample with the monomeric Aβ protein to form the reaction mix may include contacting the sample with a molar excess of the monomeric Aβ protein. The molar excess may be greater than a total molar amount of Aβ protein monomer included in the soluble, misfolded Aβ protein in the sample. The monomeric Aβ protein and/or the soluble, misfolded Aβ protein may include one or more peptides formed via β- or γ-secretase cleavage of amyloid precursor protein. The monomeric Aβ protein and/or the soluble, misfolded Aβ protein may include one or more of: Abeta40 and Abeta42.

In various embodiments of the methods described herein, the soluble, misfolded Aβ protein may substantially be the soluble, misfolded Aβ aggregate. The amplified portion of misfolded Aβ protein may substantially be one or more of: the amplified portion of the soluble, misfolded Aβ aggregate and the insoluble, misfolded Aβ aggregate. The monomeric, folded Aβ protein may be produced by one of: chemical synthesis, recombinant production, or extraction from non-recombinant biological samples.

As used herein, “αS” or “alpha-synuclein” refers to full-length, 140 amino acid α-synuclein protein, e.g., “αS-140.” Other isoforms or fragments may include “αS-126,” alpha-synuclein-126, which lacks residues 41-54, e.g., due to loss of exon 3; and “αS-112” alpha-synuclein-112, which lacks residue 103-130, e.g., due to loss of exon 5. The αS may be present in brains of individuals suffering from PD or suspected of having PD. Various αS isoforms may include and are not limited to αS-140, αS-126, and αS-112. Various αS peptides may be associated with neuronal damage associated with PD.

In embodiments where the misfolded protein and the misfolding protein substrate may correspond to αS, the methods may include determining a presence, absence, or amount of a soluble, misfolded αS protein in a sample. As described herein, methods and kits for determining a presence of a soluble, misfolded αS protein in a sample may be effective to determine an absence of the soluble, misfolded αS protein in the sample. The soluble, misfolded αS protein described herein may be a pathogenic protein, e.g., causing or leading to various neural pathologies associated with PD or other disorders associated with αS misfolding, aggregation or deposition. The methods may include contacting the sample with a monomeric, αS protein to form an incubation mixture. The methods may include conducting an incubation cycle two or more times on the incubation mixture effective to form an amplified portion of misfolded αS protein from the monomeric αS protein. Each incubation cycle may include incubating the incubation mixture effective to cause misfolding and/or aggregation of at least a portion of the monomeric αS protein in the presence of the soluble, misfolded αS protein, e.g., to form an amplified portion of misfolded αS protein. Each incubation cycle may include physically disrupting the incubation mixture effective to at least partly de-aggregate at least a portion of a misfolded αS aggregate present e.g., to release the soluble, misfolded αS protein. The methods may include determining the presence, absence, or amount of the soluble, misfolded αS protein in the sample by detecting at least a portion of the soluble, misfolded αS protein. The soluble, misfolded αS protein may include one or more of: a soluble, misfolded αS monomer and a soluble, misfolded αS aggregate. The amplified portion of misfolded αS protein may include one or more of: an amplified portion of the soluble, misfolded αS monomer, an amplified portion of the soluble, misfolded αS aggregate, and an insoluble, misfolded αS aggregate.

As used herein, “monomeric αS protein” refers to single αS protein molecules in their native, nonpathogenic configuration. “Soluble, misfolded αS protein” refers to misfolded oligomers or aggregates of αS protein that remain in solution. Examples of soluble, misfolded αS protein may include any number of aggregated misfolded αS protein monomers so long as the misfolded αS protein remains soluble. For example, soluble, misfolded αS protein may include aggregates of between 2 and about 50 units of misfolded αS protein monomer. In some examples, aggregates may be referred to as oligomers or polymers. In some examples, aggregation may be referred to as oligomerization or polymerization.

Soluble, misfolded αS protein may aggregate or oligomerize to form insoluble aggregates and/or higher oligomers, leading to αS protein aggregates in the form of protofibrils, fibrils, and eventually plaques or inclusion bodies. “Seeds” or “nuclei” refer to misfolded αS protein or short fragmented fibrils, particularly soluble, misfolded αS protein, with catalytic activity for inducing further misfolding, oligomerization, and/or aggregation. Such nucleation-dependent polymerization may be characterized by a slow lag phase wherein aggregated nuclei may form, which may then catalyze rapid formation of further and/or larger aggregates. The lag phase may be minimized or removed by addition of pre-formed nuclei or seeds. In some examples, “seeds” or “nuclei” may exclude unaggregated monomers of αS protein. Without wishing to be bound by theory, it is believed that at least under some conditions, monomeric, misfolded αS protein may not be stable, and the minimum stable size of pathogenic, misfolded αS protein may be an aggregate of two monomer units of misfolded αS protein.

As used herein, aggregates of αS protein refer to non-covalent associations of protein including soluble, misfolded αS protein. Aggregates of αS protein may be “de-aggregated”, broken up, or disrupted to release smaller aggregates, e.g., soluble, misfolded αS protein and fragmented fibrils. The catalytic activity of a collection of misfolded αS protein aggregate seeds may scale, at least in part with the number of seeds in a mixture. Accordingly, disruption of aggregates of αS protein in a mixture to release soluble, misfolded αS protein and fragmented fibrils seeds may lead to an increase in catalytic activity for aggregation of monomeric αS protein.

In various embodiments, methods for determining a presence, absence, or amount of a soluble, misfolded αS protein in a sample are provided. The methods may include contacting the sample with Thioflavin T and a molar excess of a monomeric αS protein to form an incubation mixture. The molar excess may be greater than an amount of αS protein monomer included in the soluble, misfolded αS protein in the sample. The methods may include conducting an incubation cycle two or more times to form the incubation mixture into a detection mixture. Each incubation cycle may include incubating the incubation mixture effective to cause misfolding and/or aggregation of at least a portion of the monomeric αS protein in the presence of the soluble, misfolded αS protein to form an amplified portion of misfolded αS protein. Each incubation cycle may include shaking the incubation mixture effective to at least partly de-aggregate at least a portion of a misfolded αS aggregate present, e.g., to release the soluble, misfolded αS protein. The methods may also include determining the presence, absence, or amount of the soluble, misfolded αS protein in the sample by detecting a fluorescence of the Thioflavin T corresponding to soluble, misfolded αS protein. The soluble, misfolded αS protein may include one or more of: a soluble, misfolded αS monomer and a soluble, misfolded αS aggregate. The amplified portion of misfolded αS protein may include one or more of: an amplified portion of the soluble, misfolded αS monomer, an amplified portion of the soluble, misfolded αS aggregate, and an insoluble, misfolded αS aggregate.

In various embodiments, methods for determining a presence, absence, or amount of a soluble, misfolded αS protein in a sample are provided. The methods may include capturing soluble, misfolded αS protein from the sample. The methods may include contacting the captured soluble, misfolded αS protein with a molar excess of monomeric αS protein to form an incubation mixture. The molar excess may be greater than an amount of αS protein monomer included in the captured soluble, misfolded αS protein. The methods may include conducting an incubation cycle two or more times to form the incubation mixture into a detection mixture. Each incubation cycle may include incubating the incubation mixture effective to cause misfolding and/or aggregation of at least a portion of the monomeric αS protein in the presence of the captured soluble, misfolded αS protein to form an amplified portion of misfolded αS protein. Each incubation cycle may include physically disrupting the incubation mixture effective to at least partly de-aggregate at least a portion of a misfolded αS aggregate present, e.g., to release the soluble, misfolded αS protein. The methods may also include determining the presence of the soluble, misfolded αS protein in the sample by detecting at least a portion of the soluble, misfolded αS protein. The soluble, misfolded αS protein may include one or more of: a soluble, misfolded αS monomer and a soluble, misfolded αS aggregate. The captured, soluble, misfolded αS protein may include one or more of: a captured, soluble, misfolded αS monomer and a captured, soluble, misfolded αS aggregate. The amplified portion of misfolded αS protein may include one or more of: an amplified portion of the soluble, misfolded αS monomer, an amplified portion of the soluble, misfolded αS aggregate, and an insoluble, misfolded αS aggregate.

As used herein, references to the soluble, misfolded αS protein may include any form of the soluble, misfolded αS protein, distributed in the sample, the incubation mixture, the detection mixture, and the like. For example, references to the soluble, misfolded αS protein may include the soluble, misfolded αS protein, for example, the soluble, misfolded αS protein in a sample from a subject suffering from PD. References to the soluble, misfolded αS protein may include, for example, the amplified portion of misfolded αS protein, e.g., in the incubation mixture and/or the detection mixture. References to the soluble, misfolded αS protein may include the captured soluble, misfolded αS protein, e.g., soluble, misfolded αS protein captured from the sample using αS specific antibodies.

In some embodiments, the incubation mixture may include the monomeric αS protein in a concentration, or in a concentration range, of one or more of: between about 1 nM and about 2 mM; between about 10 nM and about 200 μM; between about 100 nM and about 20 μM; or between about 1 μM and about 10 μM; and about 7 μM.

In several embodiments, the sample may be taken from a subject. The method may include determining or diagnosing the presence of PD in the subject according to the presence, absence, or amount of the soluble, misfolded αS protein in the sample. The presence, absence, or amount of the soluble, misfolded αS protein in the sample may be determined compared to a control sample taken from a control subject. The method may include determining or diagnosing the presence of a disease associated with alpha-synuclein homeostasis in the subject according to the presence, absence, or amount of the soluble, misfolded αS protein in the sample. The method may include determining or diagnosing the presence of Multiple System Atrophy in the subject according to the presence, absence, or amount of the soluble, misfolded αS protein in the sample.

In various embodiments, the detecting may include detecting an amount of the soluble, misfolded αS protein in the sample. The method may include determining or diagnosing the presence of PD in the subject by comparing the amount of the soluble, misfolded αS protein in the sample to a predetermined threshold amount.

In several embodiments, the sample may be taken from a subject exhibiting no clinical signs of dementia according to cognitive testing. The method may include determining or diagnosing the presence of PD in the subject according to the presence, absence, or amount of the soluble, misfolded αS protein in the sample.

In various embodiments, the sample may be taken from a subject exhibiting clinical signs of dementia according to cognitive testing. The method may further include determining or diagnosing the presence of PD as a contributing factor to the clinical signs of dementia in the subject according to the presence, absence, or amount of the soluble, misfolded αS protein in the sample.

In several embodiments, the method may include taking the sample from the subject. The subject may be one of a: human, mouse, rat, dog, cat, cattle, horse, deer, elk, sheep, goat, pig, or non-human primate. Non-human animals may be wild or domesticated. The subject may be one or more of: at risk of PD, having PD, under treatment for PD; at risk of having a disease associated with dysregulation, misfolding, aggregation or disposition of αS; such as Multiple System Atrophy; having a disease associated with dysregulation, misfolding, aggregation or disposition of αS; under treatment for a disease associated with dysregulation, misfolding, aggregation or disposition of αS; and the like.

In various embodiments, the method may include determining or diagnosing a progression or homeostasis of PD in the subject by comparing the amount of the soluble, misfolded αS protein in the sample to an amount of the soluble, misfolded αS protein in a comparison sample taken from the subject at a different time compared to the sample.

For example, several novel therapeutics that are targeting αS homeostasis through various mechanisms are currently under development. Therapeutic approaches targeting αS homeostasis may include active immunization, such as PD01A+ or PD03A+, or passive immunization such as PRX002. A PMCA assay for αS oligomers may be employed to determine which patients may be treated with an αS modulating therapy. Patients showing a change, e.g, increase or decrease, in the level of αS oligomers as detected by the PMCA method may be classified as “responders” to αS modulating therapy, and may be treated with a therapeutic reducing the levels of αS oligomers. Patients lacking an aberrant αS homeostasis may be classified as “non responders” and may not be treated. Patients who could benefit from therapies aimed at modulating αS homeostasis may thus be identified.

Further, for example, the amount of αS oligomers may be measured in samples from patients using PMCA. Patients with elevated αS measurements may be treated with therapeutics modulating αS homeostasis. Patients with normal αS measurements may not be treated. A response of a patient to therapies aimed at modulating αS homeostasis may be followed. For example, αS oligomer levels may be measured in a patient sample at the beginning of a therapeutic intervention. Following treatment of the patient for a clinical meaningful period of time, another patient sample may be obtained and αS oligomer levels may be measured. Patients who show a change in αS levels following therapeutic intervention may be considered to respond to the treatment. Patients who show unchanged αS levels may be considered non-responding. The methods may include detection of αS aggregates in patient samples containing components that may interfere with the PMCA reaction.

In some embodiments, the subject may be treated with an αS modulating therapy. The method may include comparing the amount of the soluble, misfolded αS protein in the sample to an amount of the soluble, misfolded αS protein in a comparison sample. The sample and the comparison sample may be taken from the subject at different times over a period of time under the αS modulating therapy. The method may include determining or diagnosing the subject is one of: responsive to the αS modulating therapy according to a change in the soluble, misfolded αS protein over the period of time, or non-responsive to the αS modulating therapy according to homeostasis of the soluble, misfolded αS protein over the period of time. The method may include treating the subject determined to be responsive to the αS modulating therapy with the αS modulating therapy. The αS modulating therapy may include inhibiting the production of αS, inhibiting the aggregation of αS, e.g., with a suitable inhibitor, active or passive immunotherapy approaches, and the like.

In several embodiments, the amount of αS oligomers may be measured in samples from patients using PMCA. Patients with elevated αS measurements may be treated with disease modifying therapies for PD. Patients with normal αS measurements may not be treated. A response of a patient to disease-modifying therapies may be followed. For example, αS oligomer levels may be measured in a patient sample at the beginning of a therapeutic intervention. Following treatment of the patient for a clinical meaningful period of time, another patient sample may be obtained and αS oligomer levels may be measured. Patients who show a change in αS levels following therapeutic intervention may be considered to respond to the treatment. Patients who show unchanged αS levels may be considered non-responding. The method may include comparing the amount of the soluble, misfolded αS protein in the sample to an amount of the soluble, misfolded αS protein in a comparison sample. The sample and the comparison sample may be taken from the subject at different times over a period of time under the disease-modifying therapy for PD. The method may include determining the subject is one of: responsive to the disease-modifying therapy for PD according to a change in the soluble, misfolded αS protein over the period of time, or non-responsive to the disease-modifying therapy for PD according to homeostasis of the soluble, misfolded αS protein over the period of time. The method may include treating the subject determined to be responsive to the disease-modifying therapy for PD with the disease-modifying therapy for PD. Disease-modifying therapies of PD may include GDNF (Glia cell-line derived neurotrophic factor), inosine, Calcium-channel blockers, specifically Cav1.3 channel blockers such as isradipine, nicotine and nicotine-receptor agonists, GM-CSF, glutathione, PPAR-gamma agonists such as pioglitazone, and dopamine receptor agonists, including D2/D3 dopamine receptor agonists and LRRK2 (leucine-rich repeat kinase 2) inhibitors.

The methods may include detection of αS aggregates in patient samples containing components that may interfere with the PMCA reaction.

In several embodiments, the method may include selectively concentrating the soluble, misfolded αS protein in one or more of the sample, the incubation mixture, and the detection mixture. The selectively concentrating the soluble, misfolded αS protein may include pre-treating the sample prior to forming the incubation mixture. The selectively concentrating the soluble, misfolded αS protein may include pre-treating the incubation mixture prior to incubating the incubation mixture. The selectively concentrating the soluble, misfolded αS protein may include contacting one or more αS specific antibodies to the soluble, misfolded αS protein to form a captured soluble, misfolded αS protein. The one or more αS specific antibodies may include one or more of: α/β-synuclein N-19; α-synuclein C-20-R; α-synuclein 211; α-synuclein Syn 204; α-synuclein 2B2D1; α-synuclein LB 509; α-synuclein SPM451; α-synuclein 3G282; α-synuclein 3H2897; α/β-synuclein Syn 202; α/β-synuclein 3B6; α/β/γ-synuclein FL-140; and the like. The one or more αS specific antibodies may include one or more of: α/β-synuclein N-19; α-synuclein C-20-R; α-synuclein 211; α-synuclein Syn 204; and the like. Such antibodies may be obtained as follows: α/β-synuclein N-19 (cat. No. SC-7012, Santa Cruz Biotech, Dallas, Tex.); α-synuclein C-20-R (SC-7011-R); α-synuclein 211 (SC-12767); α-synuclein Syn 204 (SC-32280); α-synuclein 2B2D1 (SC-53955); α-synuclein LB 509 (SC-58480); α-synuclein SPM451 (SC-52979); α-synuclein 3G282 (SC-69978); α-synuclein 3H2897 (SC-69977); α/β-synuclein Syn 202 (SC-32281); α/β-synuclein 3B6 (SC-69699); and α/β/γ-synuclein FL-140 (SC-10717). The one or more αS specific antibodies may include one or more of: an antibody specific for an amino acid sequence of αS and an antibody specific for a conformation of the soluble, misfolded αS protein. The one or more αS specific antibodies may be coupled to a solid phase. The solid phase may include one or more of a magnetic bead and a multiwell plate.

For example, ELISA plates may be coated with the antibodies used to capture αS from the patient sample. The antibody-coated ELISA plates may be incubated with a patient sample, unbound materials may be washed off, and the PMCA reaction may be performed. Antibodies may also be coupled to beads. The beads may be incubated with the patient sample and used to separate αS-antibody complexes from the remainder of the patient sample.

In various embodiments, the contacting the sample with the monomeric αS protein to form the incubation mixture may include contacting a molar excess of the monomeric αS protein to the sample including the captured soluble, misfolded αS protein. The molar excess of the monomeric αS protein may be greater than a total molar amount of αS protein monomer included in the captured soluble, misfolded αS protein. The incubating the incubation mixture may be effective to cause misfolding and/or aggregation of at least a portion of the monomeric αS protein in the presence of the captured soluble, misfolded αS protein to form the amplified portion of misfolded αS protein.

In some embodiments, the protein aggregate may include one or more of: the monomeric αS protein, the soluble, misfolded αS protein, and a captured form of the soluble, misfolded αS protein.

In several embodiments, contacting the sample with the monomeric αS protein to form the incubation mixture may be conducted under physiological conditions. Contacting the sample with the monomeric αS protein to form the incubation mixture may include contacting the sample with a molar excess of the monomeric αS protein. The molar excess may be greater than a total molar amount of αS protein monomer included in the soluble, misfolded αS protein in the sample. The monomeric αS protein and/or the soluble, misfolded αS protein may include one or more peptides formed via proteolytic cleavage of αS-140. The monomeric αS protein and/or the soluble, misfolded αS protein may include one or more of: αS-140, αS-126, αS-112, and the like. As used herein, “αS-140” refers to full-length, 140 amino acid α-synuclein protein. Other isoforms may include “αS-126,” alpha-synuclein-126, which lacks residues 41-54, e.g., due to loss of exon 3; and “αS-112” alpha-synuclein-112, which lacks residue 103-130, e.g., due to loss of exon 5.

In various embodiments of the methods described herein, the soluble, misfolded αS protein may substantially be the soluble, misfolded αS aggregate. The amplified portion of misfolded αS protein may substantially be one or more of: the amplified portion of the soluble, misfolded αS aggregate and the insoluble, misfolded αS aggregate. The monomeric αS protein may be produced by one of: chemical synthesis, recombinant production, or extraction from non-recombinant biological samples.

As used herein, “tau” refers to proteins are the product of alternative splicing from a single gene, e.g., MAβ T (microtubule-associated protein tau) in humans. Tau proteins include up to full-length and truncated forms of any of tau's isoforms. Various isoforms include, but are not limited to, the six tau isoforms known to exist in human brain tissue, which correspond to alternative splicing in exons 2, 3, and 10 of the tau gene. Three isoforms have three binding domains and the other three have four binding domains. Misfolded tau may be present in brains of individuals suffering from AD or suspected of having AD, or other tauopathies that, like AD, regard misfolding in the presence of both 4R and 3R tau isoforms. Misfolded tau may also be present in diseases that regard misfolding of primarily 4R tau isoforms, such as progressive supranuclear palsy (PSP), tau-dependent frontotemporal dementia (FTD), corticobasal degeneration (CBD), mild cognitive impairment (MCI), argyrophilic grain disease (AgD), and the like.

In various embodiments, a method is provided for determining a presence or absence in a sample of misfolded tau, e.g., 4R tau, or determining an amount of the misfolded tau in the sample using the calibration curve as described herein for the misfolded protein. The method may include performing a protein misfolding cyclic amplification (PMCA) procedure. The PMCA procedure may include forming a reaction mix by contacting the sample with the misfolding substrate protein, e.g., native 4R tau. The PMCA procedure may include conducting an incubation cycle two or more times under conditions effective to form the misfolded protein, e.g., misfolded 4R tau. Each incubation cycle may include incubating the reaction mix effective to cause misfolding and/or aggregation of the misfolding substrate protein in the presence of the misfolded protein. Each incubation cycle may include disrupting the reaction mix effective to form the amplified misfolded protein. The PMCA procedure may include determining the presence or absence in the sample of the misfolded protein by analyzing the reaction mix for the presence or absence of the amplified misfolded protein. The misfolded protein may include the misfolding substrate protein. The amplified misfolded protein may include the misfolding substrate protein.

In various embodiments, a method is provided for determining a presence or absence in a subject of a tauopathy corresponding to a misfolded protein, corresponding to determining a presence or absence in a sample of misfolded tau, e.g., 4R tau, or determining an amount of the misfolded tau in the sample using the calibration curve as described herein for the misfolded protein. The method may include providing a sample from the subject. The method may include performing at least a PMCA procedure. The PMCA procedure may include forming a reaction mix by contacting a portion of the sample with a misfolding substrate protein. The misfolding substrate protein may include a tau isoform. The misfolding substrate protein may be subject to pathological misfolding and/or aggregation in vivo to form the misfolded protein. The PMCA procedure may include conducting an incubation cycle two or more times under conditions effective to form an amplified misfolded protein. Each incubation cycle may include incubating the reaction mix effective to cause misfolding and/or aggregation of the misfolding substrate protein in the presence of the misfolded protein. Each incubation cycle may include disrupting the reaction mix effective to form the amplified misfolded protein. The PMCA procedure may include determining the presence or absence in the sample of the misfolded protein by analyzing the reaction mix for the presence or absence of the amplified misfolded protein. The PMCA procedure may include determining the presence or absence of the tauopathy in the subject according the presence or absence of the misfolded protein in the sample. The misfolded protein may include the misfolding substrate protein. The amplified misfolded protein may include the misfolding substrate protein. The method may provide that the tauopathy excludes Pick's disease when the misfolding substrate protein consists of monomeric 3R tau.

In various embodiments, the misfolding substrate protein may independently include a tau isoform, e.g., 3R tau, 4R tau, and the like. In several embodiments, the misfolding substrate protein may include 4R tau. The misfolding substrate protein may include 3R tau. The misfolding substrate protein may exclude 3R tau, for example, when the sample corresponds to Pick's disease or is drawn from a subject having Pick's disease. The misfolding substrate protein may be soluble. The misfolding substrate protein may be monomeric. The misfolding substrate protein may be in a native in vivo conformation.

In some embodiments the sample may be taken from a subject. The method may include determining or diagnosing the presence or absence of a tauopathy in the subject according to the presence or absence of the misfolded protein in the sample.

In various embodiments, the tauopathy may include a primary tauopathy or a secondary tauopathy. The tauopathy may be characterized at least in part by misfolding and/or aggregation of 4R tau protein. The tauopathy may be characterized at least in part by misfolding and/or aggregation of 4R tau protein and 3R tau protein. The tauopathy may be characterized at least in part by misfolded and/or aggregated 4R tau protein, in a ratio to misfolded and/or aggregated 3R tau protein, of one of about: 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, and 99:1, or a range between any two of the preceding ratios, for example, between 1:99 and 99:1.

In several embodiments, the methods may include characterizing an identity of the tauopathy by analyzing the amplified misfolded protein or one or more corresponding PMCA kinetic parameters thereof for a signature of at least one of: Alzheimer's disease (AD), Parkinson's Disease (PD), Progressive Supranuclear Palsy (PSP), FrontoTemporal Dementia (FTD), Corticobasal degeneration (CBD), Mild cognitive impairment (MCI), Argyrophilic grain disease (AgD) Traumatic Brain Injury (TBI), Chronic Traumatic Encephalopathy (CTE), and Dementia Pugilistica (DP). For example, characterizing the identity of the tauopathy may include determining the one or more corresponding PMCA kinetic parameters, including one or more of: lag phase, T₅₀, amplification rate, and amplification extent. Characterizing the identity of the tauopathy may include comparing the one or more corresponding PMCA kinetic parameters to one or more corresponding predetermined corresponding PMCA kinetic parameters that are characteristic of the identity of the tauopathy to determine a similarity or difference effective to characterize the identity of the tauopathy.

In various embodiments, the methods are provided such that the tauopathy specifically excludes Pick's disease. In various embodiments, the exclusion of Pick's disease does not encompass the remainder of Pick's complex of diseases.

In several embodiments, the methods may include determining or diagnosing the presence or absence of a tauopathy in the subject including comparing the presence or absence of the misfolded protein in the sample to a control sample taken from a control subject. The detecting may include detecting an amount of the misfolded protein in the sample. The sample may be taken from a subject. The methods may include determining or diagnosing the presence or absence of a tauopathy in the subject by comparing the amount of the misfolded protein in the sample to a predetermined threshold amount. The sample may be taken from a subject exhibiting no clinical signs of dementia according to cognitive testing. The methods may include determining or diagnosing the presence or absence of a tauopathy in the subject according to the presence or absence of the misfolded protein in the sample. The sample may be taken from a subject exhibiting no cortex plaques or tangles according to contrast imaging. The methods may include determining or diagnosing the presence or absence of a tauopathy in the subject according to the presence or absence of the misfolded protein in the sample. The sample may be taken from a subject exhibiting clinical signs of dementia according to cognitive testing. The methods may include determining or diagnosing the presence or absence of a tauopathy as a contributing factor to the clinical signs of dementia in the subject according to the presence or absence of the misfolded protein in the sample. The sample may be taken from a subject exhibiting no clinical signs of dementia according to cognitive testing. The subject may exhibit a predisposition to dementia according to genetic testing. The genetic testing may indicate, for example, an increased risk of tauopathy according to one or two copies of the ApoE4 allele, variants of the brain derived neurotrophic factor (BDNF) gene, such as the va166met allele, in which valine at AA position 66 is replaced by methionine, and the like. The methods may include determining or diagnosing the presence or absence of a tauopathy in the subject according to the presence or absence of the misfolded protein in the sample.

The subject may be one or more of: at risk of a tauopathy, having the tauopathy, and under treatment for the tauopathy. The methods may include determining a progression or homeostasis of a tauopathy in the subject by comparing the amount of the misfolded protein in the sample to an amount of the misfolded protein in a comparison sample taken from the subject at a different time compared to the sample. The subject may be treated with a tauopathy modulating therapy. The methods may include comparing the amount of the misfolded protein in the sample to an amount of the misfolded protein in a comparison sample. The sample and the comparison sample may be taken from the subject at different times over a period of time under the tauopathy modulating therapy. The methods may include determining the subject is one of: responsive to the tauopathy modulating therapy according to a change in the misfolded protein over the period of time, or non-responsive to the tauopathy modulating therapy according to homeostasis of the misfolded protein over the period of time. The methods may include treating the subject determined to be responsive to the tauopathy modulating therapy with the tauopathy modulating therapy. The methods may include treating the subject with a tauopathy modulating therapy to inhibit production of the misfolding substrate protein or to inhibit aggregation of the misfolded protein.

In some embodiments, the subject may be treated with a protein misfolding disorder (PMD) modulating therapy. The method may include comparing the amount of the each misfolded protein aggregate in the sample to an amount of the each misfolded protein aggregate in a comparison sample. The sample and the comparison sample may be taken from the subject at different times over a period of time under the each misfolded protein aggregate modulating therapy. The method may include determining or diagnosing the subject is one of: responsive to the each misfolded protein aggregate modulating therapy according to a change in the each misfolded protein aggregate over the period of time, or non-responsive to the each misfolded protein aggregate modulating therapy according to homeostasis of the each misfolded protein aggregate over the period of time. The method may include treating the subject determined to be responsive to the each misfolded protein aggregate modulating therapy with the each misfolded protein aggregate modulating therapy. For AD, for example, the PMD modulating therapy may include administration of one or more of: an inhibitor of BACE1 (beta-secretase 1); an inhibitor of γ-secretase; and a modulator of Aβ homeostasis, e.g., an immunotherapeutic modulator of Aβ homeostasis. The Aβ modulating therapy may include administration of one or more of: E2609; MK-8931; LY2886721; AZD3293; semagacestat (LY-450139); avagacestat (BMS-708163); solanezumab; crenezumab; bapineuzumab; BIIB037; CAD106; 8F5 or 5598 or other antibodies raised against Aβ globulomers, e.g., as described by Barghorn et al, “Globular amyloid β-peptide₁₋₄₂ oligomer-a homogenous and stable neuropathological protein in Alzheimer's disease” J. Neurochem., 2005, 95, 834-847, the entire teachings of which are incorporated herein by reference; ACC-001; V950; Affitrope AD02; and the like.

For PD, for example, the PMD modulating therapy may include active immunization, such as PD01A+ or PD03A+, passive immunization such as PRX002, and the like. The PMD modulating therapy may also include treatment with GDNF (Glia cell-line derived neurotrophic factor), inosine, Calcium-channel blockers, specifically Cav1.3 channel blockers such as isradipine, nicotine and nicotine-receptor agonists, GM-CSF, glutathione, PPAR-gamma agonists such as pioglitazone, and dopamine receptor agonists, including D2/D3 dopamine receptor agonists and LRRK2 (leucine-rich repeat kinase 2) inhibitors.

In several embodiments, the amount of misfolded protein may be measured in samples from patients using PMCA. Patients with elevated misfolded protein measurements may be treated with disease modifying therapies for a PMD. Patients with normal misfolded protein measurements may not be treated. A response of a patient to disease-modifying therapies may be followed. For example, misfolded protein levels may be measured in a patient sample at the beginning of a therapeutic intervention. Following treatment of the patient for a clinical meaningful period of time, another patient sample may be obtained and misfolded protein levels may be measured. Patients who show a change in misfolded protein levels following therapeutic intervention may be considered to respond to the treatment. Patients who show unchanged misfolded protein levels may be considered non-responding. The methods may include detection of misfolded protein aggregates in patient samples containing components that may interfere with the PMCA reaction.

In some embodiments, the methods may provide that that the tauopathy is not primarily characterized by misfolding and/or aggregation of 3R tau protein. For example, the tauopathy may be characterized at least in part by misfolded and/or aggregated 4R tau protein, in a ratio to misfolded and/or aggregated 3R tau protein, of one of about: 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, and 99:1, or a range between any two of the preceding ratios, for example, between 1:99 and 99:1.

In one embodiment, a calibration curve may be determined. For example, a plurality of stock solutions may be prepared, each having a known concentration of PrP^{Sc}. The stock solutions are separately mixed with a first PrP^{C} source to form separate stock reaction mixes. The stock reaction mixes are incubated and subjected to sonication. The incubation and sonication steps may be repeated a plurality of times, until a PrP^{Sc} signal may be detected (by, e.g., western blotting) for each stock reaction mix. The concentration of each stock solution may be compared with the number of times the incubation and sonication steps were repeated, to determine a calibration curve.

In one particular embodiment, PrP^{Sc} was partially purified by precipitation in the presence of sarkosyl. This partially purified PrP^{Sc} was used as a stock solution in buffer. To estimate the PrP^{Sc} concentration in the stock solution, partially purified PrP^{Sc} in the stock solution was subjected to deglycosylation and, subsequently, to western blot assay. To determine the PrP^{Sc} concentration of the stock solution, the stock solution western blot assay signal was compared to western blot and enzyme-linked immunosorbent assay signals of known concentrations of PrP^{Sc}.

Once the PrP^{Sc} concentration of the stock solution was estimated, the stock solution was diluted and separated into several sub-solutions having various known PrP^{Sc} concentrations ranging from 1×10⁻⁸ to 1×10⁻¹⁹ g. The sub-solutions were separately spiked into separate normal hamster brain homogenates to form stock reaction mixes. The stock reaction mixes were subjected to serial rounds of PMCA cycles. In one particular embodiment, one “round” of PMCA cycles corresponded to 144 cycles. More or fewer than 144 cycles may also be used. The number of PMCA rounds required to produce a signal detectable by western blot was determined.

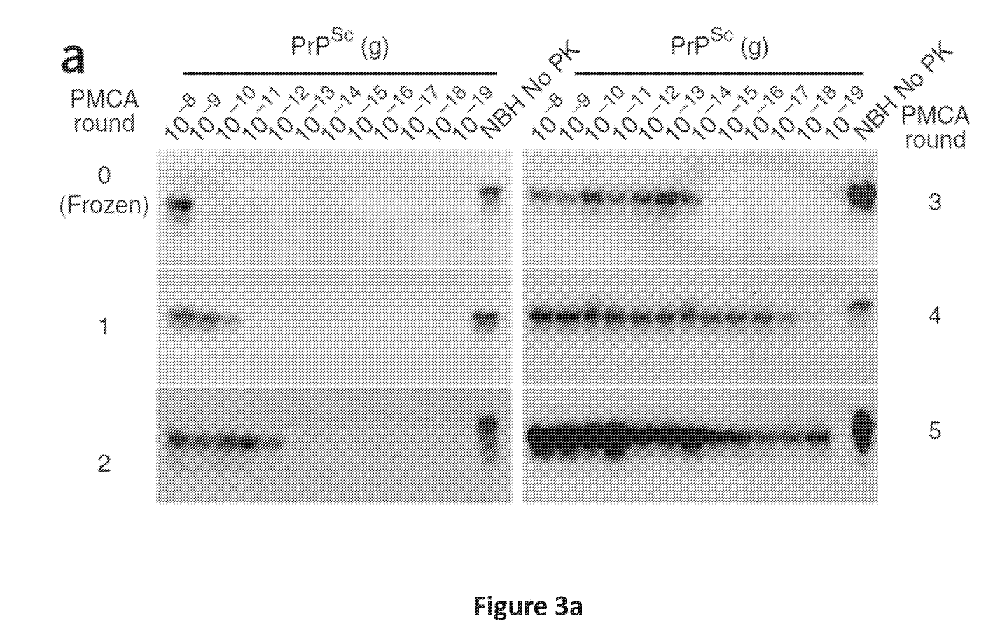

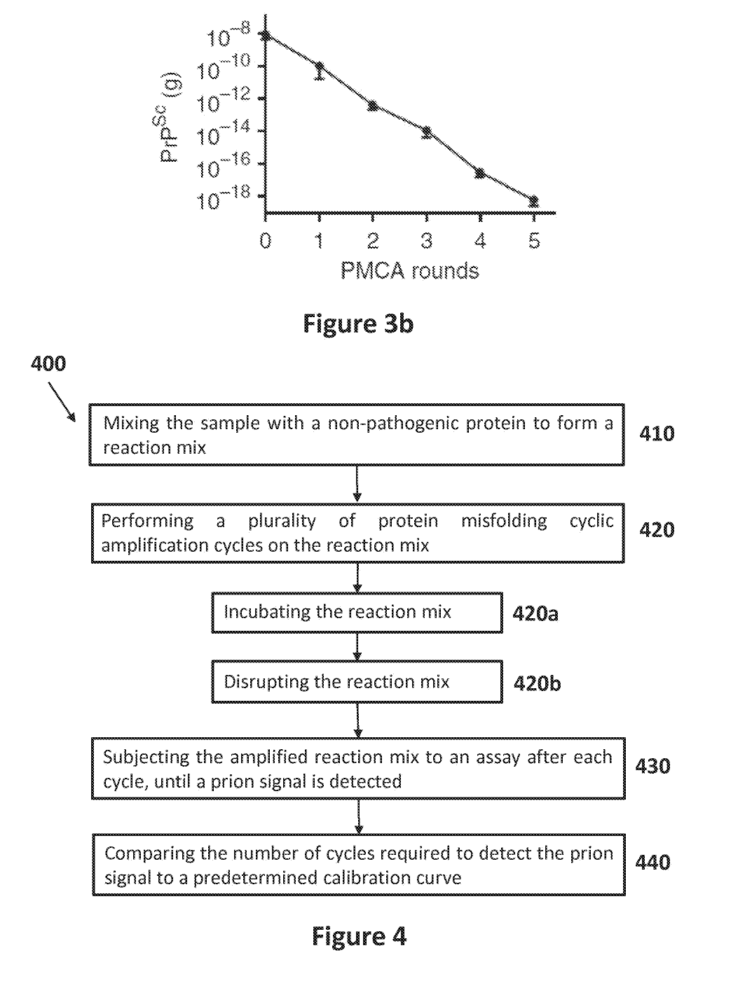

FIG. 3a illustrates western blot assays (3F4 antibody) of the stock reaction mixes. All samples except the normal brain homogenate (NBH) used as a migration control were digested with proteinase K (PK).

The concentrations of the sub-solutions were plotted against the number of PMCA rounds required for detection, to provide a calibration curve. The results are shown in FIG. 3b. More particularly, the western blots from FIG. 3a were analyzed by densitometry, and the last detectable signal after each PMCA round was plotted, yielding a standard calibration curve to estimate PrP^{Sc} concentrations.

It was determined that there may be a direct relationship between the quantity of PrP^{Sc} in a given sample and the number of PMCA cycles corresponding to detection of the quantity of PrP^{Sc} in the sample. By extrapolating the number of PMCA rounds corresponding to detection in an unknown sample, the concentration of PrP^{Sc} in the sample may be estimated.

Thus, in one embodiment, an unknown sample may be subjected to a PrP^{C} source to form a sample reaction mix. The sample reaction mix is incubated and subjected to sonication. The incubation and sonication steps may be repeated a plurality of times, until a PrP^{Sc} signal is detected for the sample reaction mix. The number of times the incubation and sonication steps were repeated is compared to the predetermined calibration curve to determine the concentration of PrP^{Sc} in the sample.

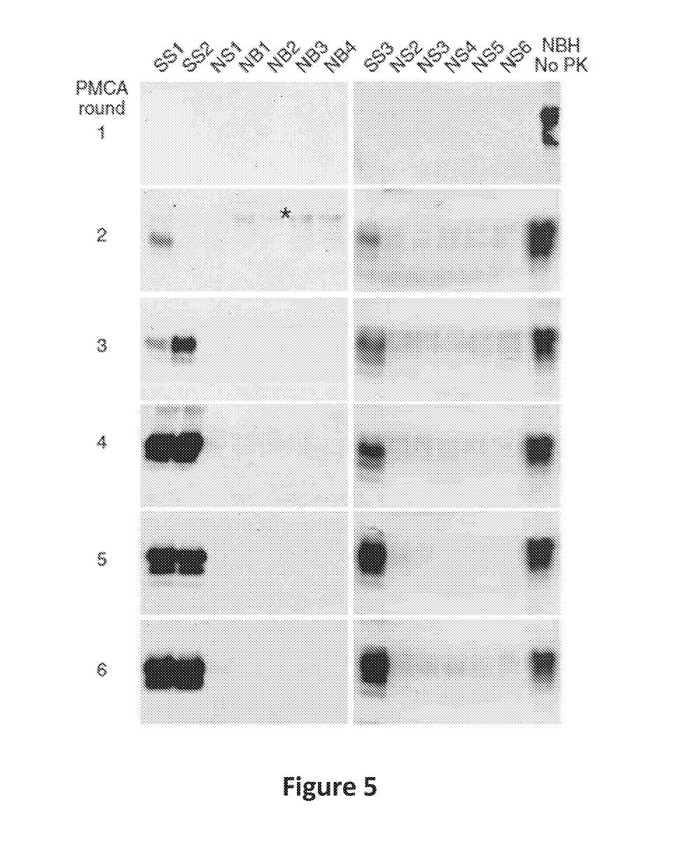

In one embodiment, as depicted in FIG. 4, where a calibration curve is known and provided, e.g., as a part of a kit, a method 400 for estimating the concentration of prion in a sample may comprise:

mixing the sample with a non-pathogenic protein to form a reaction mix (step 410);

performing a plurality of protein misfolding cyclic amplification cycles on the reaction mix (step 420), each cycle comprising:
incubating the reaction mix (sub-step 420a); and
disrupting the reaction mix (sub-step 420b);

subjecting the amplified reaction mix to an assay after each cycle, until a prion signal is detected (step 430); and

comparing the number of cycles required to detect the prion signal to a predetermined calibration curve (step 440).

In one embodiment, a kit for detecting and quantifying prion in a sample is provided, the kit comprising:
(a) a non-pathogenic protein source;
(b) a sonicator; and
(c) a calibration curve.

As used herein, a “misfolded protein” or “misfolded protein aggregate” is a protein that contains in part or in full a structural conformation of the protein that differs from the structural conformation that exists when involved in its typical, non-pathogenic normal function within a biological system. A misfolded protein may aggregate. A misfolded protein may localize in a protein aggregate. A misfolded protein may be a non-functional protein. A misfolded protein may be a pathogenic conformer of the protein. Monomeric protein compositions may be provided in native, nonpathogenic conformations without the catalytic activity for misfolding, oligomerization, and aggregation associated with seeds (a misfolded protein oligomer capable of catalyzing misfolding under PMCA conditions). Monomeric protein compositions may be provided in seed-free form.

As used herein, “monomeric protein” refers to single protein molecules. “Soluble, aggregated misfolded protein” refers to oligomers or aggregations of monomeric protein that remain in solution. Examples of soluble, misfolded protein may include any number of protein monomers so long as the misfolded protein remains soluble. For example, soluble, misfolded protein may include monomers or aggregates of between 2 and about 50 units of monomeric protein.

Monomeric and/or soluble, misfolded protein may aggregate to form insoluble aggregates, higher oligomers, and/or tau fibrils. For example, aggregation of Aβ or tau protein may lead to protofibrils, fibrils, and eventually misfolded plaques or tangles that may be observed in AD or tauopathy subjects. “Seeds” or “nuclei” refer to misfolded protein or short fragmented fibrils, particularly soluble, misfolded protein with catalytic activity for further misfolding, oligomerization, and aggregation. Such nucleation-dependent aggregation may be characterized by a slow lag phase wherein aggregate nuclei may form, which may then catalyze rapid formation of further aggregates and larger oligomers and polymers. The lag phase may be minimized or removed by addition of pre-formed nuclei or seeds. Monomeric protein compositions may be provided without the catalytic activity for misfolding and aggregation associated with misfolded seeds. Monomeric protein compositions may be provided in seed-free form.

As used herein, “soluble” species may form a solution in biological fluids under physiological conditions, whereas “insoluble” species may be present as precipitates, fibrils, deposits, tangles, or other non-dissolved forms in such biological fluids under physiological conditions. Such biological fluids may include, for example, fluids, or fluids expressed from one or more of: amniotic fluid; bile; blood; cerebrospinal fluid; cerumen; skin; exudate; feces; gastric fluid; lymph; milk; mucus, e.g. nasal secretions; mucosal membrane, e.g., nasal mucosal membrane; peritoneal fluid; plasma; pleural fluid; pus; saliva; sebum; semen; sweat; synovial fluid; tears; urine; and the like. Insoluble species may include, for example, fibrils of Aβ, αS, 4R tau, 3R tau, combinations thereof such as 3R tau+4R tau, and the like. A species that dissolves in a non-biological fluid but not one of the aforementioned biological fluids under physiological conditions may be considered insoluble. For example, fibrils of Aβ, αS, 4R tau, 3R tau, combinations thereof such as 3R tau +4R tau, and the like may be dissolved in a solution of, e.g., a surfactant such as sodium dodecyl sulfate (SDS) in water, but may still be insoluble in one or more of the mentioned biological fluids under physiological conditions.

In some embodiments, the sample may exclude insoluble species of the misfolded proteins, e.g., Aβ, αS and/or tau as a precipitate, fibril, deposit, tangle, plaque, or other form that may be insoluble in one or more of the described biological fluids under physiological conditions.

For example, the sample may exclude the misfolded protein in fibril form. The sample may exclude misfolded proteins in insoluble form, e.g., the sample may exclude the misfolded proteins as precipitates, fibrils, deposits, tangles, plaques, or other insoluble forms, e.g., in fibril form. The methods described herein may include preparing the sample by excluding the misfolded proteins in insoluble form, e.g., by excluding from the sample the misfolded proteins as precipitates, fibrils, deposits, tangles, plaques, or other insoluble forms, e.g., in fibril form. The kits described herein may include instructions directing a user to prepare the sample by excluding from the sample the misfolded proteins as precipitates, fibrils, deposits, tangles, plaques, or other insoluble forms, e.g., in fibril form. The exclusion of such insoluble forms of the described misfolded proteins from the sample may be substantial or complete.

In some embodiments, the sample may exclude insoluble species of the misfolded proteins such as Aβ, αS, 4R tau, 3R tau, combinations thereof such as 3R tau+4R tau and the like as a precipitate, fibril, deposit, tangle, plaque, or other form that may be insoluble in one or more of the described biological fluids under physiological conditions.

For example, in some embodiments, the sample may exclude tau in fibril form. The sample may exclude misfolded tau proteins in insoluble form, e.g., the sample may exclude the misfolded tau proteins as precipitates, fibrils, deposits, tangles, plaques, or other insoluble forms, e.g., in fibril form. The methods described herein may include preparing the sample by excluding the misfolded protein in insoluble form, e.g., by excluding from the sample the misfolded tau protein as precipitates, fibrils, deposits, tangles, plaques, or other insoluble forms, e.g., in fibril form. The kits described herein may include instructions directing a user to prepare the sample by excluding from the sample the misfolded tau protein as precipitates, fibrils, deposits, tangles, plaques, or other insoluble forms, e.g., in fibril form. The exclusion of such insoluble forms of the described misfolded proteins from the sample may be substantial or complete.

As used herein, aggregates of misfolded protein refer to non-covalent associations of protein including soluble, misfolded protein. Aggregates of misfolded protein may be “de-aggregated”, or disrupted to break up or release soluble, misfolded protein. The catalytic activity of a collection of soluble, misfolded protein seeds may scale, at least in part with the number of such seeds in a mixture. Accordingly, disruption of aggregates of misfolded protein in a mixture to release misfolded protein seeds may lead to an increase in catalytic activity for oligomerization or aggregation of monomeric protein.

In some embodiments, the methods may include preparing the reaction mix characterized by at least one concentration of: the misfolding substrate protein, e.g., 4R tau, of less than about 20 μM; heparin of less than about 75 μM; NaCl of less than about 190 mM; and Thioflavin T of less than about 9.5 μM.

In various embodiments, the reaction mix may include the misfolding substrate protein at a concentration in μM of one or more of about: 0.001, 0.01, 0.1, 0.25, 0.5, 0.75, 1, 1.25, 1,5, 1.75, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 50, 70, 100, 150, 200, 250, 500, 750, 1000, 1500, or 2000, or a range between any two of the preceding values, for example, between about 0.001 μM and about 2000 μM.

The reaction mix may include heparin at a concentration in μM of one or more of about: 0.001, 0.01, 0.1, 0.25, 0.5, 0.75, 1, 1.25, 1,5, 1.75, 2, 2.5, 3, 3.5, 4, 4.5, 5, 6, 7, 8, 9, 10 11, 12, 12.5, 15, 17.5, 20, 25, 30, 35, 40, 45, 50, 55, 60, 65, 70, and 75, or a range between any two of the preceding values, for example, between about 0.001 μM and about 75 μM. For example, the reaction mix may include heparin when the misfolded protein and the misfolding substrate protein correspond to tau, e.g., 4R tau.

The reaction mix may include a buffer composition of one or more of: Tris-HCL, PBS, MES, PIPES, MOPS, BES, TES, and HEPES. The methods may include preparing the reaction mix including the buffer composition at a total concentration of one or more of about: 1 μM, 10 μM, 100 μM, 250 μM, 500 μM, 750 μM, 1 mM, 10 mM, 100 mM, 250 mM, 500 mM, 750 mM, and 1M, or a range between any two of the preceding values, for example, between about 1 μM and about 1 M.

The reaction mix may include a salt composition at a total concentration of one or more of: 1 μM, 10 μM, 100 μM, 250 μM, 500 μM, 750 μM, 1 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 250 mM, 500 mM, 750 mM, and 1M, or a range between any two of the preceding values, for example, between about 1 μM and about 1 M. The salt composition may include one or more of: NaCl and KCl.

In various embodiments, the reaction mix may be characterized by a pH of one or more of about: 5, 5.5, 6, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, or 9, or a range between any two of the preceding values, e.g., from about pH 5 to about pH 9.

In some embodiments, the reaction mix may include an indicator at a total concentration of one or more of: 1 nM, 10 nM, 100 nM, 250 nM, 500 nM, 750 nM, 1 μM, 2 μM, 3 μM, 4 μM, 5 μM, 6 μM, 7 μM, 8 μM, 9 μM, 9.5 μM, 10 μM, 25 μM, 50 μM, 100 μM, 250 μM, 500 μM, 750 μM, 1 mM, or a range between any two of the preceding values, for example, between about 1 nM and about 1 mM.

In some embodiments, the incubating may include heating or maintaining the reaction mix ata temperature in ° C. of one of: 5, 10, 15, 20, 22.5, 25, 27.5, 30, 31, 32, 33, 34, 35, 36, 37, 38, 39, 40, 41, 42, 43, 44, 45, 46, 47, 50, 55, 60, or a range between any two of the preceding values, for example, between about 5° C. and about 60° C.

In several embodiments, an indicator of the misfolded protein may be contacted to the reaction mix. The indicator of the misfolded protein may be characterized by an indicating state in the presence of the misfolded protein and a non-indicating state in the absence of the misfolded protein. Determining the presence or amount of the misfolded protein in the sample may include detecting the indicating state of the indicator of the misfolded protein. The indicating state of the indicator and the non-indicating state of the indicator may be characterized by a difference in fluorescence. Determining the presence, absence, or amount of the misfolded protein in the sample may include detecting the difference in fluorescence. The methods may include contacting a molar excess of the indicator of the misfolded protein to the reaction mix. The molar excess may be greater than a total molar amount of protein monomer included in the misfolding substrate protein and the misfolded protein in the reaction mix. The indicator of the misfolded protein may include one or more of: a thioflavin, e.g., thioflavin T or thioflavin S; Congo Red, m-I-Stilbene, Chrysamine G, PIB, BF-227, X-34, TZDM, FDDNP, MeO-X-04, IMPY, NIAD-4, luminescent conjugated polythiophenes, a fusion with a fluorescent protein such as green fluorescent protein and yellow fluorescent protein, derivatives thereof, and the like.

In various embodiments, the method may include determining an amount of the misfolded protein in the sample. For example, known amounts of in vitro, synthetic misfolded protein aggregate seeds may be added to various portions of a biological fluid of a healthy patient, e.g., CSF. Subsequently, PMCA may be performed on the various portions. In each of the various portions, a fluorescent indicator of the misfolded protein aggregate may be added, and fluorescence may be measured as a function of, e.g., number of PMCA cycles, to determine various PMCA kinetics parameters, e.g., number of PMCA cycles to maximum fluorescence signal, number of PMCA cycles to 50% of maximum fluorescence signal, lag phase in increase of fluorescence signal, rate of increase in fluorescence signal versus PMCA cycles, and the like. A calibration curve showing the relationship between the concentration of synthetic seeds added and the PMCA kinetic parameters. The kinetic parameters may be measured for unknown samples and compared to the calibration curve to determine the expected amount of seeds present in a particular sample. Alternatively, the amount of the misfolded protein in the sample may be determined by a series of known dilutions of the sample, and PMCA of each serial dilution to determine whether the misfolded protein can be detected or not in a particular dilution. The amount of the misfolded protein in the undiluted sample can be estimated based on the known dilution that results in no detection of the misfolded protein by PMCA. In another example, the amount of the misfolded protein in the sample may be determined by a series of known dilutions of the sample, and PMCA to determine a detection signal in each serial dilution. The collected detection signals in the serial dilutions can be fit, e.g., via least squares analysis, to determine whether the misfolded protein can be detected or not in a particular dilution. In another example, the amount of the misfolded protein in the sample may be determined by known amounts of antibodies to the misfolded protein. The amount of the misfolded protein may be determined according to the methods described herein using the calibration curve. The amount determined using two or more of the above methods may be compared.

In some embodiments, the methods may include detecting the amount of the misfolded protein in the sample at a sensitivity of at least about one or more of: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%, e.g., at least about 70%. The methods may include detecting the amount of the misfolded protein in the sample at less than about one or more of: 625, 62.5, 6.25, 630 μg, 63 μg, 6.3 μg, 630 ng, 63 ng, 6.3 ng, 630 pg, 200 pg, 63 pg, 6.3 pg, 630 fg, 300 fg, 200 fg, 125 fg, 63 fg, 50 fg, 30 fg, 15 fg, 12.5 fg, 10 fg,5 fg, or 2.5 fg, The methods may include detecting the amount of the misfolded protein in the sample at less than about one or more of: 100 nmol, 10 nmol, 1 nmol, 100 pmol, 10 pmol, 1 pmol, 100 fmol, 10 fmol, 3 fmol, 1 fmol, 100 attomol, 10 attomol, 5 attomol, 2 attomol, 1 attomol, 0.75 attomol, 0.5 attomol, 0.25 attomol, 0.2 attomol, 0.15 attomol, 0.1 attomol, and 0.05 attomol, e.g., less than about 100 nmol. The methods may include detecting the amount of the misfolded protein in the sample in a molar ratio to the misfolding substrate protein included by the sample. The molar ratio may be less than about one or more of: 1: 100, 1: 10,000, 1: 100,000, and 1: 1,000,000, e.g., less than about 1: 100. The methods may include determining the amount of the misfolded protein in the sample compared to a control sample.

In several embodiments, the methods may include detecting the misfolded protein in the sample with a specificity of at least about one or more of: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%, e.g. at least about 70%.

The methods may include detecting the misfolded protein including one or more of: a Western Blot assay, a dot blot assay, an enzyme-linked immunosorbent assay (ELISA), a fluorescent protein/peptide binding assay, a thioflavin binding assay, a Congo Red binding assay, a sedimentation assay, electron microscopy, atomic force microscopy, surface plasmon resonance, and spectroscopy. The ELISA may include a two-sided sandwich ELISA. The spectroscopy may include one or more of: quasi-light scattering spectroscopy, multispectral ultraviolet spectroscopy, confocal dual-color fluorescence correlation spectroscopy, Fourier-transform infrared spectroscopy, capillary electrophoresis with spectroscopic detection, electron spin resonance spectroscopy, nuclear magnetic resonance spectroscopy, and Fluorescence Resonance Energy Transfer (FRET) spectroscopy. Detecting the misfolded protein may include contacting the reaction mix with a protease; and detecting the misfolded protein using anti-misfolded protein antibodies or antibodies specific for a misfolded protein in one or more of: a Western Blot assay, a dot blot assay, and an ELISA.

In various embodiments, the misfolding substrate protein may be provided in labeled form. The misfolding substrate protein in labeled form may include one or more of: a covalently incorporated radioactive amino acid, a covalently incorporated, isotopically labeled amino acid, and a covalently incorporated fluorophore. The methods may include detecting the misfolding substrate protein in labeled form as incorporated into the amplified misfolded protein.

In some embodiments, the sample may include one or more of a bio-fluid, e.g., blood, a biomaterial, e.g., cerumen, a homogenized tissue, and a cell lysate. The sample may include one or more of: amniotic fluid; bile; blood; cerebrospinal fluid; cerumen; skin; exudate; feces; gastric fluid; lymph; milk; mucus; mucosal membrane; peritoneal fluid; plasma; pleural fluid; pus; saliva; sebum; semen; sweat; synovial fluid; tears; and urine. The sample may be derived from cells or tissue of one or more of: skin, brain, heart, liver, pancreas, lung, kidney, gastro-intestine, nerve, mucous membrane, blood cell, gland, and muscle. The tissue or cells may be homogenized, lysed, or otherwise extracted by conventional methods. The methods may include obtaining the sample from a subject, such as by drawing a bio-fluid or biomaterial, performing a tissue biopsy, and the like. The volume of each portion of the sample added to a particular PMCA reaction, e,g., in fluid or homogenized form, may be a volume in μL of one of about 5,000, 4,000, 3,000, 2,000, 1000, 900, 800, 750, 700, 650, 600, 550, 500, 450, 400, 350, 300, 250, 200, 150, 125, 100, 90, 80, 70, 60, 50, 40, 30, 25, 20, 15, 10, 5, or 1, or a range between any two of the preceding values, e.g., from about 1 μL to about 1000 μL. In some embodiments, when the sample is CSF, the amount of each portion added to a particular PMCA reaction may be a volume in μL of any of the preceding, for example, one of about 80, 70, 60, 50, 40, 30, 25, 20, 15, or 10, or a range between any two of the preceding values, e.g., e.g., from about 10 μL to about 80 μL, e.g., about 40 μL. In some embodiments, when the sample is plasma, the amount of each portion added to a particular PMCA reaction may be a volume in μL of any of the preceding, for example, one of about 750, 700, 650, 600, 550, 500, 450, 400, 350, 300, 250, or a range between any two of the preceding values, e.g., e.g., from about 250 μL to about 750 μL, e.g., about 500 μL. In some embodiments, when the sample is blood, the amount of each portion added to a particular PMCA reaction may be a volume in μL of any of the preceding, for example, one of about 5,000, 4,000, 3,000, 2,000, 1000, 900, 800, 750, 700, 650, 600, 550, 500, 450, 400, 350, 300, 250, or 200, or a range between any two of the preceding values, e.g., from about 200 μL to about 1000 μL.

### EXAMPLES

### Example 1

### Sample Preparation

Syrian hamsters were intraperitoneally inoculated with 263,000 prions and monitored for the appearance of clinical symptoms, using a standard scale known in the art. When disease was confirmed, urine was collected using metabolic cages. The hamsters were then killed by CO₂ inhalation, and brains, spleens, and blood were collected.

Brain and spleen homogenates were prepared at 10% (wt/vol) in PBS plus Complete cocktail of protease inhibitors (Boehringer Mannheim). The samples were clarified by a 45 s low speed centrifugation. Blood samples were obtained directly from the heart in tubes containing citrate. Plasma and buffy coat were separated by centrifugation in ficoll gradient. Samples of normal brain homogenate used for PMCA substrate were obtained after perfusing hamsters with PBS and 5 mM EDTA. Solutions of 10% normal brain homogenate were made in conversion buffer (PBS without Ca²⁺ and Mg²⁺ with 150 mM NaCl, 1.0% triton X-100, and Complete protease inhibitors). Debris was removed by a 45 s low speed centrifugation in an Eppendorf centrifuge.

### Example 2

### PrP^{Sc} Partial Purification by Sarkosyl Precipitation

To minimize interference in PMCA from other components present in tissues and fluids, PrP^{Sc} was partially enriched by sarkosyl precipitation. More particularly, samples were incubated with one volume of 20% sarkosyl for 10 min at room temperature and centrifuged at 100,000 g for 1 h at 4° C. Supernatants were discarded and pellets were re-suspended into two volumes of 10% sarkosyl. The centrifugation process was repeated, and pellets were re-suspended directly in 10% normal brain homogenate prepared in conversion buffer. Following this protocol, PrP^{Sc} was recovered in the pellet fraction at greater than 90% yield.

### Example 3

### PMCA procedure

Samples were loaded onto 0.2 mL PCR tubes. Tubes were positioned on an adaptor placed on a plate holder of a microsonicator (Misonix model 4000), and samples were subjected to cycles of 30 min incubation at 37° C., followed by a 20 s pulse of sonication set at a potency of 7.5 (75%). Samples were incubated, without shaking, immersed in the water of the sonicator bath. Standard PMCA rounds included 144 cycles. After each round of cycles, a 10 μL aliquot of the amplified material was diluted into 90 μL of normal brain homogenate and a new round of PMCA cycles was performed.

### Example 4

### PrP^{Sc} Detection

Samples were digested with 50 μg mL⁻¹ of PK at 37° C. for 1 h, and the reaction was stopped by adding NuPAGE LDS sample buffer. The proteins were fractionated using 4-12% SDS-PAGE, electroblotted into Hybond ECL nitrocellulose membrane, and probed with the 3F4 antibody (Covance) (dilution 1:5,000). The immunoreactive bands were visualized by ECL Plus western blotting detection system and quantified by densitometry using a UVP Bioimaging System EC3 apparatus.

### Example 5

### Detection of PrPsc in the Spleen of Scrapie-Affected Hamsters

As described in Example 1, samples of brain, spleen, blood, and urine were collected from five hamsters exhibiting clinical signs of disease after intraperitoneal inoculation with 263,000 prions. As described in Example 2, the PrP^{Sc} was partially purified by sarkosyl precipitation to remove components that may affect PMCA efficiency. After centrifugation, PrP^{Sc} pellets were re-suspended directly into healthy hamster brain homogenate and subjected to serial rounds of 144 PMCA cycles.

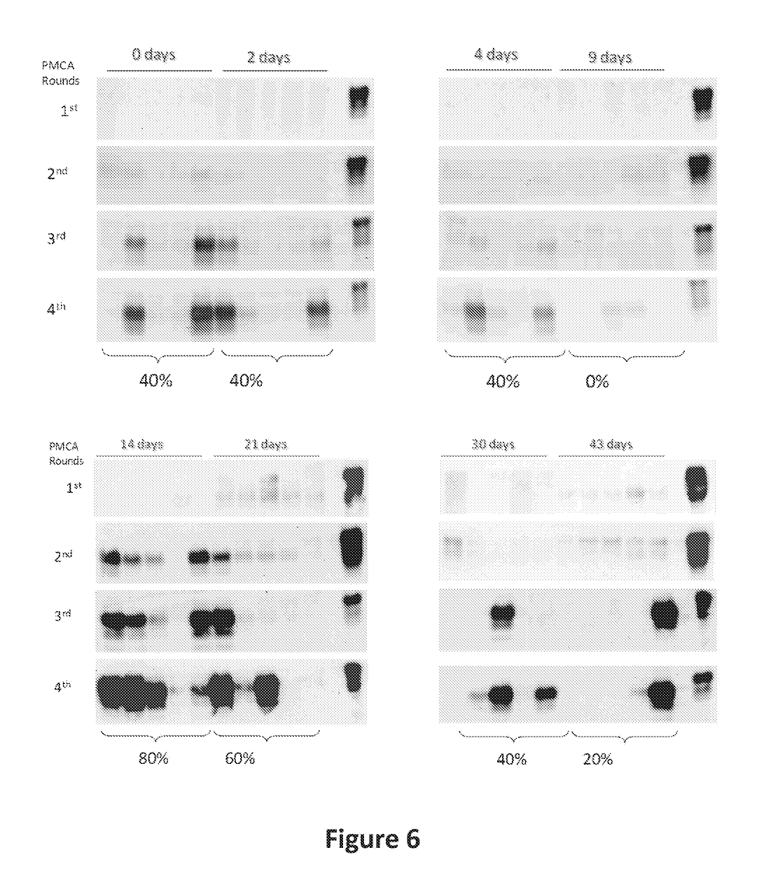

Three spleen samples were positive for prion disease. Of those three, PrP^{Sc} was detectable after two rounds of PMCA for two samples and after the third round for the third sample. FIG. 5 illustrates western blot assays of PrP^{Sc}-affected hamster spleen suspended in normal hamster brain homogenate and subjected to serial PMCA. The three scrapie spleen samples are labeled SS1, SS2, and SS3.

With further reference to FIG. 5, control samples of normal (i.e., non-infected) spleen homogenate (samples NS1-NS6) and brain homogenate (samples NB1-NB4) were subjected to the same PMCA procedure to assess the rate of spontaneous appearance of PrP^{Sc} reactivity. Normal brain homogenate (NBH) not digested with PK was used as a migration control. No PrP^{Sc} signal was detected after six rounds of PMCA in any of the control samples.

Extrapolation from the calibration curve of FIG. 3b provides that the average concentration of PrP^{Sc} in the symptomatic spleen was 20 pg g⁻¹. PrP^{Sc} concentrations in other tissues and fluids were also analyzed. The results are shown in Table 1:

TABLE 1
PrP^{Sc} Concentration in Scrapie-Affected Hamsters

| Source | PrP^{Sc} Concentration in Tissues (g/g) and fluids (g/mL) |
|---|---|
| Brain | 2.3 × 10⁻⁵ ± 6.8 × 10⁻⁶ |
| Spleen | 2.0 × 10⁻¹¹ ± 1.1 × 10⁻¹¹ |
| Buffy Coat | 2.6 × 10⁻¹³ ± 2.4 × 10⁻¹³ |
| Plasma | 1.3 × 10⁻¹⁴ ± 1.1 × 10⁻¹⁴ |
| Urine | 2.0 × 10⁻¹⁶ ± 1.7 × 10⁻¹⁶ |

### Example 6

### Dynamic Distribution and Quantification of PrP^{Sc} in Different Tissues and Fluids

To evaluate the application of quantitative PMCA to determine the concentration of prions in various tissues and fluids, and to understand the dynamic of PrP^{Sc} formation and accumulation in tissues and fluids at distinct stages of the disease, PrP^{Sc} levels in brains, spleens, blood fractions (plasma and buffy coat), and urine were measured at different time periods after infection.

Specifically, tissue extracts were obtained from hamsters intraperitoneally infected with 263,000 prions. Animals were sacrificed at the following time periods: 0, 2, 4, 9, 14, 21, 30, 43, 50, 71, 81, and 110 days post-inoculation. Under these conditions, animals showed the disease symptoms an average 110 days after inoculation. Samples from each of the tissues at each of the times from five different animals per group were suspended in normal hamster brain homogenate, subjected to serial rounds of PMCA, and subjected to western blotting.

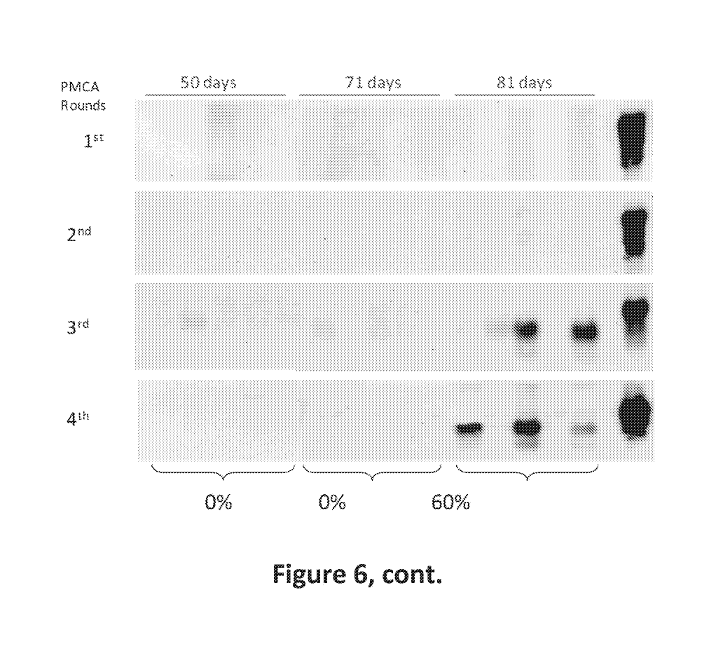

FIG. 6 illustrates western blot assays of the samples. The numbers at the top of the gels indicate the number of days after inoculation. The numbers to the left of the gels indicate the number of PMCA rounds. The numbers at the bottom of the gels indicate the percentage of PrP^{Sc}-positive animals after four rounds of PMCA.

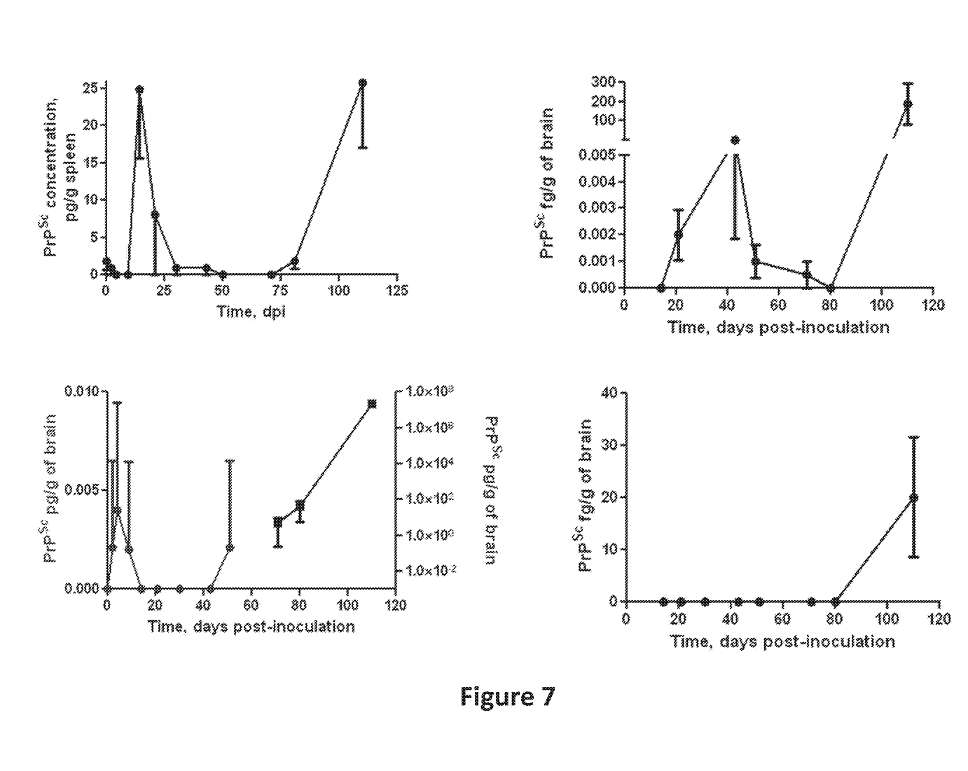

FIG. 7 illustrates plots of concentration versus the time period after inoculation for the various tissue and fluid samples. Endogenous replication of PrP^{Sc} reached high levels in spleens at early stages after infection (FIG. 7, plot A), which correlated with their presence in white blood cells (FIG. 7, plot C). Interestingly, PrP^{Sc} quantity decreased in spleens in the middle of the incubation periods, precisely prior to the time in which PrP^{Sc} began to appear in the brain (FIG. 7, plot B). The levels of PrP^{Sc} increased again in spleens close to the symptomatic phase, to reach a quantity similar to that found in the early pre-symptomatic stage of the disease (FIG. 7, plot A). The levels of PrP^{Sc} in brains increased in an exponential way with time, starting around 50 days post-inoculation (FIG. 7, plot B). PrP^{Sc} was not detectable in brains before this time, except for a few days after inoculation, which most likely represents the influx of PrP^{Sc} present in the inoculum across the blood brain barrier. The quantities of PrP^{Sc} estimated in brains two to nine days after inoculation reached around 2-4 fg/g of brain (FIG. 7, plot A). This quantity is probably not enough to trigger prion replication and is likely eliminated by the normal clearance mechanisms. The later re-appearance of PrP^{Sc} in the brains likely means a more constant influx of prions produced by peripheral replication and transport through the peripheral nerves. The biphasic behavior of PrP^{Sc} in spleens is similar to that expected in the blood buffy coat fraction, which mostly contains white cells. However, the quantities of PrP^{Sc} in buffy coat are three orders of magnitude lower than those measured in spleens (FIG. 7, plot C). In plasma, PrP^{Sc} was only detectable at or close to the symptomatic phase of the disease (FIG. 7, plot D), and the quantities are around 10 times lower than in the buffy coat fraction.

These findings indicate that the presence of PrP^{Sc} in blood may have two different sources: peripheral replication in the spleen at early stages and brain leakage at late stages. Prions in blood at the pre-symptomatic phase are restricted to the white cells, which likely were coming from cells previously resident in the spleen. At the symptomatic phase, cerebral prions are likely leaking to the blood and circulate in a cell-free manner in plasma and possibly produce a second wave of spleen infection.

A comparison of the estimated quantities of PrP^{Sc} in the organs and fluids tested at the symptomatic phase reveals that the quantity in the brain is 106, 108, and 109 times higher than in spleen, buffy coat, and plasma, respectively, in this particular model (Table 2). However, at half of the incubation period (50 days post inoculation) the quantity of prions in the brain is only around 2 fg/g, which represents only 3- and 2000-times higher than spleen and buffy coat (Table 2).

TABLE 2
Estimated PrP^{Sc} Concentrations (g/g tissue or g/mL of fluid) in Different Tissues
and Biological Fluids at Distinct Time Periods After Inoculation

| | | Late Pre- | Mid Pre- | Early Pre- |
|---|---|---|---|---|
| | Symptomatic | Symptomatic | Symptomatic | Symptomatic |
| Source | (110 dpi) | (80 dpi) | (51 dpi) | (21 dpi) |
| Brain | 2.3 × 10⁻⁵ ± 6.8 × | 5.1 × 10⁻¹¹ ± 4.8 × | 2.2 × 10⁻¹⁵ ± 2.0 × | Not detectable |
| | 10⁻⁶ | 10⁻¹¹ | 10⁻¹⁵ | |
| Spleen | 2.0 × 10⁻¹¹ ± 1.1 × | 5.2 × 10⁻¹³ ± 6.8 × | 1.6 × 10⁻¹⁶ ± 1.2 × | 8.0 × 10⁻¹² ± 7.1 × |
| | 10⁻¹¹ | 10⁻¹³ | 10⁻¹⁶ | 10⁻¹² |
| Buffy Coat | 1.1 × 10⁻¹³ ± 0.9 × | Not detectable | 1.0 × 10⁻¹⁸ ± 1.0 × | 1.9 × 10⁻¹⁸ ± 1.2 × |
| | 10⁻¹³ | | 10⁻¹⁸ | 10⁻¹⁸ |
| Plasma | 5.2 × 10⁻¹⁵ ± 3.1 × | Not detectable | Not detectable | Not detectable |
| | 10⁻¹⁵ | | | |
| Urine | 2.0 × 10⁻¹⁶ ± 1.7 × | Not done | Not done | Not done |
| | 10⁻¹⁶ | | | |

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B), it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.”

While the present application has been illustrated by the description of particular embodiments, and while the embodiments have been described in considerable detail, it is not an intention to restrict or in any way limit the scope of the appended claims to such detail. With the benefit of the present application, additional advantages and modifications will readily appear to those skilled in the art. Therefore, the application, in its broader aspects, is not limited to the specific details and illustrative examples shown and described. Accordingly, departures may be made from such details without departing from the spirit or scope of the general inventive concept.

What is claimed is:

1. A method for preparing a calibration curve useful for quantitatively estimating a concentration of a misfolded protein in a sample, the method comprising:
preparing a plurality of stock solutions, each stock solution in the plurality of stock solutions having a known different concentration of the misfolded protein;
separately mixing each of the plurality of stock solutions with a misfolding protein substrate that corresponds to the misfolded protein to form a plurality of separate stock reaction mixes;
forming a plurality of separate amplified portions of the misfolded protein by:
performing a plurality of protein misfolding cyclic amplification (PMCA) cycles on each of the plurality of separate stock reaction mixes to form a plurality of separate amplified stock reaction mixes comprising the plurality of separate amplified portions of the misfolded protein, each cycle in the plurality of PMCA cycles comprising:
incubating each stock reaction mix; and
disaggregating aggregates formed in each stock reaction mix;
subjecting each of the plurality of separate amplified stock reaction mixes to an assay for a number of cycles of the plurality of PMCA cycles until a signal of the misfolded protein is detected; and
determining the calibration curve according to the known different concentration of the misfolded protein in each stock solution with the number of PMCA cycles corresponding to detection of the signal of the misfolded protein, at least a portion of the known different concentrations of the misfolded protein among the plurality of stock solutions being below a concentration detectable by the assay such that the calibration curve provides for quantitative estimation of the misfolded protein concentration in the sample below the concentration detectable by the assay,
provided that the misfolded protein and the misfolding protein substrate exclude prion protein and isoforms or conformers thereof.

2. The method of claim 1, further comprising plotting the calibration curve in the form of a standard calibration curve.

3. The method of claim 1, wherein the misfolding protein substrate is provided for mixing with each of the plurality of stock solutions in the form of one of:
a normal tissue homogenate comprising the misfolding protein substrate;
a normal biological fluid comprising the misfolding protein substrate;
the misfolding protein substrate purified from one or more of the normal tissue homogenate and the normal biological fluid;
a recombinant preparation of the misfolding protein substrate.

4. The method of claim 1, wherein the assay is one of: a western blot assay and a fluorescence assay.

5. The method of claim 1, further comprising preparing the stock reaction mixes comprising a biological fluid effective to provide the calibration curve for quantitatively estimating the concentration of the misfolded protein in a sample comprising the biological fluid, the biological fluid comprising one or more of: amniotic fluid; bile; blood; cerebrospinal fluid; cerumen; skin; exudate; feces; gastric fluid; lymph; milk; mucus; mucosal membrane; peritoneal fluid; plasma;
pleural fluid; pus; saliva; sebum; semen; sweat; synovial fluid; tears; and urine.

6. The method of claim 1, the misfolded protein and the misfolding protein substrate corresponding to one of: Aβ; αS; 3R tau; and 4R tau.

7. The method of claim 1, the misfolded protein and the misfolding protein substrate excluding 3R tau.

8. A method for quantitatively estimating a concentration of a misfolded protein in a sample, the method comprising:
mixing the sample with a misfolding protein substrate to form a reaction mix;
forming an amplified portion of the misfolded protein by:
performing a plurality of protein misfolding cyclic amplification (PMCA) cycles on the reaction mix to form an amplified reaction mix comprising the amplified portion of the misfolded protein, each cycle comprising:
incubating the reaction mix; and
disaggregating aggregates formed in the reaction mix;
subjecting the amplified reaction mix to an assay for a number of the plurality of PMCA cycles until a signal of the misfolded protein is detected; and
quantitatively estimating the concentration of the misfolded protein in the sample according to the number of PMCA cycles corresponding to detection of the signal of the misfolded protein by using a predetermined calibration curve for quantitatively estimating the concentration of the misfolded protein in the sample according to the assay, the predetermined calibration curve determined according to a plurality of known different concentrations of the misfolded protein each corresponding to a calibrating number of PMCA cycles, each calibrating number of PMCA cycles being effective to amplify each corresponding known different concentration of the misfolded protein in the presence of a misfolding protein substrate to a concentration of the misfolded protein detectable by the assay, at least a portion of the plurality of known different concentrations of the misfolded protein being below the concentration detectable by the assay such that the predetermined calibration curve provides for quantitative estimation of the misfolded protein concentration in the sample below the concentration detectable by the assay,
provided that the misfolded protein and the misfolding protein substrate exclude prion protein and isoforms or conformers thereof.

9. The method of claim 8, wherein the disaggregating comprises subjecting the reaction mix to sonication.

10. The method of claim 8, wherein the assay is one of: a western blot assay and a fluorescence assay.

11. The method of claim 8, further comprising:
removing a portion of the reaction mix;
contacting the portion with an additional portion of the misfolding protein substrate to form a second reaction mix;
performing a plurality of PMCA cycles on the second reaction mix, each cycle in the plurality of PMCA cycles comprising:
incubating the second reaction mix; and
disaggregating aggregates formed in the second reaction mix;
subjecting the disaggregated second reaction mix to an assay for a number of cycles of the plurality of PMCA cycles until the signal of the misfolded protein is detected; and
quantitatively estimating the concentration of the misfolded protein in the second reaction mix according to the number of cycles corresponding to detection of the signal of the misfolded protein by using the predetermined calibration curve.

12. The method of claim 8, the sample comprising one or more of: amniotic fluid; bile; blood; cerebrospinal fluid; cerumen; skin; exudate; feces; gastric fluid; lymph; milk; mucus; mucosal membrane; peritoneal fluid; plasma; pleural fluid; pus; saliva; sebum; semen; sweat; synovial fluid; tears; and urine.

13. The method of claim 8, quantitatively estimating the concentration of the misfolded protein in the sample comprising quantitatively estimating the concentration of the misfolded protein below the concentration detectable by the assay.

14. The method of claim 8, the misfolded protein and the misfolding protein substrate corresponding to one of: Aβ; αS; 3R tau; and 4R tau.

15. The method of claim 14, the sample comprising one or more additional misfolded and/or non-misfolded proteins different from the misfolded protein and the misfolding protein substrate.

16. The method of claim 8, provided that the misfolded protein and the misfolding protein substrate exclude 3R tau.

17. A kit for quantitatively estimating a concentration of a misfolded protein in a sample, t comprising:
a buffer solution comprising at least one misfolding protein substrate;
at least one predetermined calibration curve for quantitatively estimating the concentration of the at least one misfolded protein in the sample according to an assay, the predetermined calibration curve determined according to a plurality of known different concentrations of the misfolded protein each corresponding to a calibrating number of PMCA cycles, each calibrating number of PMCA cycles being effective to amplify each corresponding known different concentration of the misfolded protein in the presence of a misfolding protein substrate to a concentration of the misfolded protein detectable by the assay, at least a portion of the plurality of known different concentrations of the misfolded protein being below the concentration detectable by the assay such that the predetermined calibration curve provides for quantitative estimation of the misfolded protein concentration in the sample below the concentration detectable by the assay,
instructions for conducting the assay, the instructions including:
mixing the sample with the buffer solution comprising at least one misfolding protein substrate to form a reaction mix;
forming an amplified portion of the misfolded protein by:
performing a plurality of protein misfolding cyclic amplification (PMCA) cycles on the reaction mix to form an amplified reaction mix comprising the amplified portion of the misfolded protein, each cycle comprising:
incubating the reaction mix; and
disaggregating aggregates formed in the reaction mix;
subjecting the amplified reaction mix to an assay for a number of the plurality of PMCA cycles until a signal of the misfolded protein is detected; and
quantitatively estimating the concentration of the misfolded protein in the sample according to the number of PMCA cycles corresponding to detection of the signal of the misfolded protein by using a predetermined calibration curve for quantitatively estimating the concentration of the misfolded protein in the sample according to the assay.

18. The kit of claim 17, the instructions comprising disaggregating by subjecting the reaction mix to sonication.

19. The kit of claim 17, the instructions comprising conducting the assay as one of: a western blot assay and a fluorescence assay.

20. The kit of claim 17, the instructions further comprising:
removing a portion of the reaction mix;
contacting the portion with an additional portion of the buffer comprising the at least one misfolding protein substrate to form a second reaction mix;
performing a plurality of PMCA cycles on the second reaction mix, each cycle in the plurality of PMCA cycles comprising:
incubating the second reaction mix; and
disaggregating aggregates formed in the second reaction mix;
subjecting the disaggregated second reaction mix to an assay for a number of cycles of the plurality of PMCA cycles until the signal of the misfolded protein is detected; and
quantitatively estimating the concentration of the misfolded protein in the second reaction mix according to the number of cycles corresponding to detection of the signal of the misfolded protein by using the predetermined calibration curve.

21. The kit of claim 17, the instructions comprising using the sample comprising one or more of: amniotic fluid; bile; blood; cerebrospinal fluid; cerumen; skin; exudate; feces; gastric fluid; lymph; milk; mucus; mucosal membrane; peritoneal fluid; plasma; pleural fluid; pus; saliva; sebum; semen; sweat; synovial fluid; tears; and urine.

22. The kit of claim 17, the instructions comprising quantitatively estimating the concentration of the misfolded protein in the sample comprising quantitatively estimating the concentration of the misfolded protein below the concentration detectable by the assay.

23. The kit of claim 17, the buffer solution comprising at least one misfolding protein substrate selected from: Aβ; αS; 3R tau; and 4R tau.

24. The kit of claim 17, the buffer solution comprising two or more misfolding protein substrates, and the kit comprising two or more predetermined calibration curves corresponding to the two or more misfolding protein substrates.
